(19) United States
(12) Patent Application Publication
(10) Pub. No.: US 2023/0181509 A1
(43) Pub. Date: Jun. 15, 2023

(54) THE USE OF DIFFUSION ENHANCING COMPOUNDS FOR TREATMENT OF VIRAL AND BACTERIAL INDUCED RESPIRATORY DISEASE

(71) Applicant: DIFFUSION PHARMACEUTICALS LLC, Charlottesville, VA (US)

(72) Inventors: Thomas BYRNE, Alexandria, VA (US); David G. KALERGIS, Charlottesville, VA (US)

(21) Appl. No.: 17/995,220

(22) PCT Filed: Mar. 31, 2021
(86) PCT No.: PCT/US2021/025175

§ 371 Date: Sep. 30, 2022

Related U.S. Application Data
(60) Provisional application No. 63/113,140, filed on Nov. 12, 2020.
(60) Provisional application No. 63/052,893, filed on Jul. 16, 2020.
(60) Provisional application No. 63/003,841, filed on Apr. 1, 2020.
(60) Provisional application No. 63/003,259, filed on Mar. 31, 2020.

(51) Int. Cl. A61K 31/202; A61K 47/40; A61K 9/00

(52) U.S. Cl. CPC A61K 31/202; A61K 47/40; A61K 9/0019

(57) ABSTRACT

The subject invention relates to novel methods for the treatment of viral or bacterial induced respiratory disease with hypoxemia.

This application claim priority to U.S. Provisional Application No. 63/003,259 filed Mar. 31, 2020, U.S. Provisional Application No. 63/003,841 filed Apr. 1, 2020, U.S. Provisional Application No. 63/052,893 filed Jul. 16, 2020, and U.S. Provisional Application No. 63/113,140 filed Nov. 12, 2020, each of which is hereby incorporated by reference in its entirety.

### FIELD OF THE INVENTION

The subject invention relates to novel methods for the treatment of virus and bacteria induced respiratory disease with hypoxemia.

### BACKGROUND OF THE INVENTION

Carotenoids are a class of hydrocarbons consisting of isoprenoid units. The backbone of the molecule consists of conjugated carbon-carbon double and single bonds, and can have pendant groups. Carotenoids such as crocetin and trans sodium crocetinate (TSC) are known to increase the diffusivity of oxygen in water.

U.S. Pat. No. 6,060,511 relates to trans sodium crocetinate (TSC) and its uses. The patent covers various uses of TSC such as improving oxygen diffusivity and treatment of hemorrhagic shock.

U.S. Pat. No. 7,759,506 relates to synthesis methods for making bipolar trans carotenoids (BTC), including bipolar trans carotenoid salts (BTCS), and methods of using them.

U.S. Pat. No. 8,030,350 relates to improved BTC synthesis methods and novel uses of the BTC.

U.S. Pat. No. 8,293,804 relates to the use of bipolar trans carotenoids as a pretreatment and in the treatment of peripheral vascular disease.

U.S. Pat. No. 8,206,751 relates to a new class of therapeutics that enhance small molecule diffusion.

U.S. application Ser. No. 12/801,726 relates to diffusion enhancing compounds and their use alone or with thrombolytics.

There are no known efficacious treatments for pneumonia caused by SARS coronavirus, MERS coronavirus, adenovirus, hantavirus, or parainfluenza. Care is largely supportive. New methods for treating a viral or bacterial induced respiratory disease with hypoxemia are needed.

### SUMMARY OF THE INVENTION

The subject invention provides methods of treatment of human patients having, or diagnosed as having, a viral or bacterial induced respiratory disease with hypoxemia, with a diffusion enhancing compound of the invention. Included are methods of treating influenza, a corona virus infection including Covid-19, and bacterial or viral pneumonia.

Provided is a method of treating a patient in need thereof having a viral or bacterial induced respiratory disease with hypoxemia comprising administering a diffusion enhancing compound to said patient.

Further provided is a method of prophylaxis and/or treatment of hypoxemia consequent to a viral or bacterial induced respiratory disease in a patient in need thereof comprising administering a diffusion enhancing compound to said patient.

Further provided is a method of prophylaxis and/or treatment of acute respiratory distress syndrome associated with a viral or bacterial respiratory disease in a patient in need thereof comprising administering a diffusion enhancing compound to said patient.

Further provided is a method of prophylaxis and/or treatment of acute respiratory distress syndrome consequent to a viral or bacterial respiratory disease in a patient in need thereof comprising administering a diffusion enhancing compound to said patient.

Further provided is a method of prophylaxis and/or treatment of multiple organ failure consequent to a viral or bacterial respiratory disease in a patient in need thereof comprising administering a diffusion enhancing compound to said patient.

Further provided are methods of mitigation, control, and/or treatment of one or more of hypoxemia, acute respiratory distress syndrome, and multiple organ failure consequent to a viral or bacterial respiratory disease in a patient in need thereof comprising administering a diffusion enhancing compound to said patient.

Further provided is a method of improving blood oxygenation in a patient in need thereof comprising administering a diffusion enhancing compound to said patient.

Further provided is a method of treating a patient in need thereof having a viral or bacterial induced respiratory disease comprising administering a diffusion enhancing compound to said patient.

In any of the above methods, the diffusion enhancing compound is a bipolar trans carotenoid, advantageously a bipolar trans carotenoid salt (e.g., TSC). In a further embodiment, the bipolar trans carotenoid salt is formulated with a cyclodextrin. The diffusion enhancing compound is advantageously administered IV or IM. If the diffusion enhancing compound is TSC, a dose of about 0.05-5 mg/kg, e.g., 0.05-2.5 mg/kg, advantageously a dose of about 0.25-1.5 mg/kg is used. Advantageously, the diffusion enhancing compound is administered 3 days per week.

The invention also relates to a kit comprising a first vial with a diffusion enhancing compound such as TSC (which can be lyophilized), a second vial with diluent such as water for injection, and a syringe for administration. The kit may be used for any of the methods described herein.

The invention also includes a kit comprising:

a) a container comprising a diffusion enhancing compound such as TSC, and
b) instructions for using the diffusion enhancing compound to treat a patient having a viral or bacterial induced respiratory disease with hypoxemia, by administering the diffusion enhancing compound at a dose of about 0.05-5 mg/kg, e.g., 0.05-2.5 mg/kg to the patient. The kit may be used for any of the methods described herein.

Further the invention relates to a double chamber container or syringe for separately holding in the two chambers (and combining just before administration): a) a solid, in particular a lyophilizate of a diffusion enhancing compound such as TSC, and b) a liquid reconstitution medium therefor such as water for injection. The container or syringe may be used in any of the methods described herein.

### BRIEF DESCRIPTION OF THE DRAWINGS

FIG. 1 shows NEWS2 charts used for the National Early Warning Score.

### DETAILED DESCRIPTION OF THE INVENTION

The subject invention provides methods for the treatment of human patients, having, or diagnosed as having, a viral or bacterial induced respiratory disease with hypoxemia, with a diffusion enhancing compound of the invention. Included are methods of treating influenza, a corona virus infection including Covid-19, and bacterial or viral pneumonia.

Provided is a method of treating a patient (e.g., a human) in need thereof having a viral or bacterial induced respiratory disease with hypoxemia comprising administering a diffusion enhancing compound to said patient.

Further provided is a method of prophylaxis and/or treatment of hypoxemia consequent to a viral or bacterial induced respiratory disease in a patient (e.g., a human) in need thereof comprising administering a diffusion enhancing compound to said patient.

Further provided is a method of prophylaxis and/or treatment of acute respiratory distress syndrome associated with a viral or bacterial respiratory disease in a patient (e.g., a human) in need thereof comprising administering a diffusion enhancing compound to said patient.

Further provided is a method of prophylaxis and/or treatment of acute respiratory distress syndrome consequent to a viral or bacterial respiratory disease in a patient (e.g., a human) in need thereof comprising administering a diffusion enhancing compound to said patient. For instance, provided is a method of prophylaxis and/or treatment of mild acute respiratory distress syndrome consequent to a viral or bacterial respiratory disease in a patient (e.g., a human) in need thereof comprising administering a diffusion enhancing compound to said patient. For instance, provided is a method of prophylaxis and/or treatment of moderate acute respiratory distress syndrome consequent to a viral or bacterial respiratory disease in a patient (e.g., a human) in need thereof comprising administering a diffusion enhancing compound to said patient. For instance, provided is a method of prophylaxis and/or treatment of severe acute respiratory distress syndrome consequent to a viral or bacterial respiratory disease in a patient (e.g., a human) in need thereof comprising administering a diffusion enhancing compound to said patient.

Further provided is a method of prophylaxis and/or treatment of multiple organ failure consequent to a viral or bacterial respiratory disease in a patient (e.g., a human) in need thereof comprising administering a diffusion enhancing compound to said patient.

Further provided are methods of mitigation, control, and/or treatment of one or more of hypoxemia, acute respiratory distress syndrome, and multiple organ failure consequent to a viral or bacterial respiratory disease in a patient (e.g., a human) in need thereof comprising administering a diffusion enhancing compound to said patient. For instance, provided are methods of mitigation, control, and/or treatment of one or more of hypoxemia, acute respiratory distress syndrome, and multiple organ failure consequent to a viral or bacterial respiratory disease in a patient (e.g., a human) in need thereof comprising administering a diffusion enhancing compound to said patient, wherein said patient has mild acute respiratory distress syndrome, moderate acute respiratory distress syndrome, or severe acute respiratory distress syndrome.

Further provided is a method of improving blood oxygenation in a patient in need thereof comprising administering a diffusion enhancing compound to said patient.

As used herein, patient includes human and non-human. Preferably, a patient is human. In some embodiments of the methods described herein, the patient (e.g., a human) in need of treatment is a patient diagnosed with a viral or bacterial respiratory disease and at risk of developing one or more of acute respiratory distress syndrome, hypoxemia, and multiple organ failure. Patients at risk of developing one or more of acute respiratory distress syndrome, hypoxemia, and multiple organ failure include patients over age 60 (e.g., 65 or older, e.g., 70 or older, e.g., 75 or older), immunocompromised, and/or with one or more of diabetes, hypertension, congestive heart failure, chronic liver disease, and chronic renal disease.

Acute respiratory distress syndrome (ARDS) can be divided into mild ARDS (e.g., a human having 200 mm Hg<PaO₂/FiO₂≤300 mm Hg with positive end-expiratory pressure (PEEP) or continuous positive airway pressure (CPAP)≥5 cm H₂O), moderate ARDS (e.g., a human having 100 mm Hg<PaO₂/FiO₂≤200 mm Hg with positive end-expiratory pressure (PEEP)≥5 cm H₂O), and severe ARDS (e.g., a human having PaO₂/FiO₂≤100 mm Hg with positive end-expiratory pressure (PEEP)≥5 cm H₂O). If altitude is higher than 1000 m, then a correction factor should be calculated as follows: [PaO₂/FiO₂×(barometric pressure/760)].

FiO₂ is fraction of inspired oxygen. PaO₂ is arterial partial pressure of oxygen.

SpO₂/FiO₂ may be used as a non-invasive alternative to PaO₂/FiO₂.

In some embodiments of the methods disclosed herein, the patient (e.g., human) has mild ARDS, moderate ARDS, or severe ARDS or is at risk of developing mild ARDS, moderate ARDS, or severe ARDS. For instance, in some embodiments of the methods disclosed herein, the patient (e.g., human) has mild ARDS or is at risk of developing mild ARDS. Also, for instance, in some embodiments of the methods disclosed herein, the patient (e.g., human) has moderate ARDS or is at risk of developing moderate ARDS. Also, for instance, in some embodiments of the methods disclosed herein, the patient (e.g., human) has severe ARDS or is at risk of developing severe ARDS.

Patients suffering from moderate to severe COVID-19 disease present with abnormal chest radiography indicating bilateral and peripheral ground glass and consolidative opacities, with many progressing to acute respiratory distress syndrome (ARDS). These patients often go on to require transfer to an intensive care unit, mechanical ventilatory support, and possibly extra-corporeal membrane oxygenation (ECMO). COVID-19 leading to severe ARDS is associated with a high degree of morbidity and mortality. Prior to developing ARDS, COVID-19 patients may experience a period of so-called silent hypoxemia, consisting of observable hypoxemia by oxygen saturation (SaO₂) measurements, but showing minimal outward signs of respiratory distress.

In some embodiments, the methods disclosed herein result in preventing a patient from being supported with mechanical ventilation or extracorporeal membrane oxygenation.

TSC has been shown to have beneficial effects in hypoxemic situations. For example, TSC has been shown to increase whole-body oxygen consumption after hemorrhagic shock in rats. TSC increases oxygen levels in hypoxemic states, both in arterial and tissue levels, but does not in normoxic states. TSC has these effects because it increases the diffusivity of oxygen through plasma. The diffusion rate is known to be affected by both concentration and diffusivity (i.e. Fick's law).

Not only does TSC seem to enhance systemic oxygenation of tissues, but it may also affect passage of oxygen from the alveoli to erythrocytes to enhance the oxygen carrying capacity of blood in acute respiratory distress syndrome (ARDS).

A respiratory virus is a virus that can enter and invade the respiratory system, the system from the nose to lungs that allows taking in oxygen and breathing out carbon dioxide. There are many kinds of respiratory viruses such adenoviruses, rhinoviruses, respiratory syncytial viruses (RSV), influenza, and coronaviruses. Some of these viruses tend to stay in the upper respiratory tract while others may make it down to the lower respiratory tract.

The many viruses that cause the common cold including four other types of coronaviruses (OC43, HKU1, NL63, and 229E) behave very differently from the SARS-CoV2. Similarly, the SARS-CoV2 is not the same as the flu virus (influenza).

After viruses make more and more of themselves, they may invade additional cells lining the respiratory tract and begin to cause damage. Shortness of breath, chest pain or tightness, a deeper cough, and other difficulties breathing can be signs that these viruses have made it to the lower respiratory tract. These symptoms can come from inflammation of the respiratory tree, otherwise known as the bronchial tree.

Alveoli are tiny elastic air sacs within the lungs that allow oxygen and carbon dioxide to move between the lungs and bloodstream. Alveoli are also intertwined with a network of blood vessels. These blood vessels bring blood from the rest of the body that is low in oxygen and high in carbon dioxide, a waste product of metabolism. The alveoli is where oxygen from the air is exchanged with the carbon dioxide in the blood. The carbon dioxide goes into the alveoli, where it may be exhaled up through the respiratory tract and out through the nose and mouth. The blood that is newly infused with more oxygen subsequently travels to the rest of the body to provide cells with the oxygen.

If the viruses travel to the lungs and alveoli, it can become a pneumonia. As the damage to the lungs continues, acute respiratory distress syndrome (ARDS) can develop, which is when the lungs have suffered so much widespread injury that there is not enough functioning alveoli to do the gas exchange work. ARDS occurs when fluid builds up in the alveoli. The fluid keeps the lungs from filling with enough air, which means less oxygen reaches the bloodstream, which can cause hypoxemia. This deprives organs of the oxygen they need to function.

If the damage gets to the point that the lungs can no longer effectively exchange enough oxygen and carbon dioxide, respiratory failure can occur.

Coronavirus disease 2019 (COVID-19) is an infectious disease caused by severe acute respiratory syndrome coronavirus 2 (SARS-CoV-2). The disease was first identified in 2019 in Wuhan, the capital of China's Hubei province, and has since spread globally, resulting in the ongoing 2019-20 coronavirus pandemic. Common symptoms include fever, cough, and shortness of breath. Other symptoms may include muscle pain, sputum production, diarrhea, sore throat, loss of smell, and abdominal pain. While the majority of cases result in mild symptoms, some progress to pneumonia and multi-organ failure. As of March 2020, the overall rate of deaths per number of diagnosed cases is 4.6 percent; ranging from 0.2 percent to 15 percent according to age group and other health problems. In comparison, the overall mortality rate of the 1918 Spanish Flu were approximately 3% to 5%.

The virus is spread mainly through close contact and via respiratory droplets produced when people cough or sneeze. Respiratory droplets may be produced during breathing but the virus is not generally airborne. People may also contract COVID-19 by touching a contaminated surface and then their face. It is most contagious when people are symptomatic, although spread may be possible before symptoms appear. The virus can survive on surfaces up to 72 hours. Time from exposure to onset of symptoms is generally between two and fourteen days, with an average of five days. The standard method of diagnosis is by reverse transcription polymerase chain reaction (rRT-PCR) from a nasopharyngeal swab. The infection can also be diagnosed from a combination of symptoms, risk factors and a chest CT scan showing features of pneumonia.

Currently, there is no specific antiviral treatment for COVID-19. Management involves treatment of symptoms, supportive care, isolation, and experimental measures.

Those infected with the virus may be asymptomatic or develop flu-like symptoms, including fever, cough, fatigue, and shortness of breath. Emergency symptoms include difficulty breathing, persistent chest pain or pressure, confusion, difficulty waking, and bluish face or lips. Less commonly, upper respiratory symptoms, such as sneezing, runny nose, or sore throat may be seen. Symptoms such as nausea, vomiting, and diarrhea have been observed in varying percentages. Some cases in China initially presented only with chest tightness and palpitations. In March 2020 there were reports indicating that loss of the sense of smell (anosmia) may be a common symptom among those who have mild disease, although not as common as initially reported. In some, the disease may progress to pneumonia, multi-organ failure, and death. In those who develop severe symptoms, time from symptom onset to needing mechanical ventilation is typically eight days.

As is common with infections, there is a delay between the moment when a person is infected with the virus and the time when they develop symptoms. This is called the incubation period. The incubation period for COVID-19 is typically five to six days but may range from two to 14 days. 97.5% of people who develop symptoms will do so within 11.5 days of infection.

Reports indicate that not all who are infected develop symptoms, but their role in transmission is unknown. Preliminary evidence suggests asymptomatic cases may contribute to the spread of the disease. The proportion of infected people who do not display symptoms is currently unknown and being studied, with South Korea's CDC reporting that 20% of all confirmed cases remained asymptomatic during their hospital stay.

### Cause

The disease is caused by the severe acute respiratory syndrome coronavirus 2 (SARS-CoV-2). It is primarily spread between people during close contact and via respiratory droplets from coughs and sneezes.

Pathophysiology

The lungs are the organs most affected by COVID-19 because the virus accesses host cells via the enzyme ACE2, which is most abundant in the type II alveolar cells of the lungs. The virus uses a special surface glycoprotein called a “spike” (peplomer) to connect to ACE2 and enter the host cell. The density of ACE2 in each tissue correlates with the severity of the disease in that tissue and some have suggested that decreasing ACE2 activity might be protective, though another view is that increasing ACE2 using angiotensin II receptor blocker medications could be protective and that these hypotheses need to be tested. As the alveolar disease progresses, respiratory failure might develop and death may follow.

The virus also affects gastrointestinal organs as ACE2 is abundantly expressed in the glandular cells of gastric, duodenal and rectal epithelium as well as endothelial cells and enterocytes of the small intestine.

The WHO has published several testing protocols for the disease. The standard method of testing is real-time reverse transcription polymerase chain reaction (rRT-PCR). The test is typically done on respiratory samples obtained by a nasopharyngeal swab, however a nasal swab or sputum sample may also be used. Results are generally available within a few hours to two days. Blood tests can be used, but these require two blood samples taken two weeks apart and the results have little immediate value. As of 19 Mar. 2020, there were no antibody tests though efforts to develop them are ongoing. The FDA approved the first point-of-care test on 21 Mar. 2020 for use at the end of that month.

### Pathology

Few data are available about microscopic lesions and the pathophysiology of COVID-19. The main pathological findings at autopsy are:
Macroscopy: pleurisy, pericarditis, lung consolidation and pulmonary oedema
Four types of severity of viral pneumonia can be observed:
   minor pneumonia: minor serous exudation, minor fibrin exudation
   mild pneumonia: pulmonary oedema, pneumocyte hyperplasia, large atypical pneumocytes, interstitial inflammation with lymphocytic infiltration and multinucleated giant cell formation
   severe pneumonia: diffuse alveolar damage (DAD) with diffuse alveolar exudates. This diffuse DAD is responsible for the acute respiratory distress syndrome (ARDS) and severe hypoxemia observed in this disease.
   healing pneumonia: organization of exudates in alveolar cavities, and pulmonary interstitial fibrosis
   plasmocytosis in BAL
Liver: microvesicular steatosis

Influenza, commonly known as “the flu”, is an infectious disease caused by an influenza virus. Symptoms can be mild to severe. The most common symptoms include: high fever, runny nose, sore throat, muscle and joint pain, headache, coughing, and feeling tired. These symptoms typically begin two days after exposure to the virus and most last less than a week. The cough, however, may last for more than two weeks. In children, there may be diarrhea and vomiting, but these are not common in adults. Diarrhea and vomiting occur more commonly in gastroenteritis, which is an unrelated disease and sometimes inaccurately referred to as “stomach flu” or the “24-hour flu”. Complications of influenza may include viral pneumonia, secondary bacterial pneumonia, sinus infections, and worsening of previous health problems such as asthma or heart failure.

Three of the four types of influenza viruses affect humans: Type A, Type B, and Type C. Type D has not been known to infect humans, but is believed to have the potential to do so. Usually, the virus is spread through the air from coughs or sneezes. This is believed to occur mostly over relatively short distances. It can also be spread by touching surfaces contaminated by the virus and then touching the eyes, nose, or mouth. A person may be infectious to others both before and during the time they are showing symptoms. The infection may be confirmed by testing the throat, sputum, or nose for the virus. A number of rapid tests are available; however, people may still have the infection even if the results are negative. A type of polymerase chain reaction that detects the virus's RNA is more accurate.

Frequent hand washing reduces the risk of viral spread, as does wearing a surgical mask. Yearly vaccinations against influenza are recommended by the World Health Organization (WHO) for those at high risk, and by the Centers for Disease Control and Prevention (CDC) for those six months of age and older. The vaccine is usually effective against three or four types of influenza. It is usually well tolerated. A vaccine made for one year may not be useful in the following year, since the virus evolves rapidly. Antiviral drugs such as the neuraminidase inhibitor oseltamivir, among others, have been used to treat influenza.

Influenza spreads around the world in yearly outbreaks, resulting in about three to five million cases of severe illness and about 290,000 to 650,000 deaths. About 20% of unvaccinated children and 10% of unvaccinated adults are infected each year. In the northern and southern parts of the world, outbreaks occur mainly in the winter, while around the equator, outbreaks may occur at any time of the year. Death occurs mostly in high risk groups—the young, the old, and those with other health problems. Larger outbreaks known as pandemics are less frequent. In the 20th century, three influenza pandemics occurred: Spanish influenza in 1918 (17-100 million deaths), Asian influenza in 1957 (two million deaths), and Hong Kong influenza in 1968 (one million deaths). The World Health Organization declared an outbreak of a new type of influenza A/H1N1 to be a pandemic in June 2009.

Pneumonia is an inflammatory condition of the lung affecting primarily the small air sacs known as alveoli. Typically, symptoms include some combination of productive or dry cough, chest pain, fever and difficulty breathing. The severity of the condition is variable.

Pneumonia is usually caused by infection with viruses or bacteria and less commonly by other microorganisms, certain medications or conditions such as autoimmune diseases. Risk factors include cystic fibrosis, chronic obstructive pulmonary disease (COPD), asthma, diabetes, heart failure, a history of smoking, a poor ability to cough such as following a stroke and a weak immune system. Diagnosis is often based on the symptoms and physical examination. Chest X-ray, blood tests, and culture of the sputum may help confirm the diagnosis. The disease may be classified by where it was acquired, such as community- or hospital-acquired or health care-associated pneumonia.

Vaccines to prevent certain types of pneumonia are available. Other methods of prevention include hand washing and not smoking. Treatment depends on the underlying cause. Pneumonia believed to be due to bacteria is treated with antibiotics. If the pneumonia is severe, the affected person is generally hospitalized. Oxygen therapy may be used if oxygen levels are low.

Pneumonia affects approximately 450 million people globally (7% of the population) and results in about 4 million deaths per year. Pneumonia was regarded by Canadian pathologist William Osler in the 19th century as “the captain of the men of death”. With the introduction of antibiotics and vaccines in the 20th century, survival greatly improved. Nevertheless, in developing countries, and also among the very old, the very young and the chronically ill, pneumonia remains a leading cause of death.

Bacterial and viral cases of pneumonia usually result in similar symptoms. Some causes are associated with classic, but non-specific, clinical characteristics. Viral pneumonia presents more commonly with wheezing than bacterial pneumonia. Pneumonia was historically divided into “typical” and “atypical” based on the belief that the presentation predicted the underlying cause. However, evidence has not supported this distinction, therefore it is no longer emphasized.

Pneumonia is due to infections caused primarily by bacteria or viruses and less commonly by fungi and parasites. Although there are over 100 strains of infectious agents identified, only a few are responsible for the majority of the cases. Mixed infections with both viruses and bacteria may occur in roughly 45% of infections in children and 15% of infections in adults. A causative agent may not be isolated in approximately half of cases despite careful testing.

The term pneumonia is sometimes more broadly applied to any condition resulting in inflammation of the lungs (caused for example by autoimmune diseases, chemical burns or drug reactions); however, this inflammation is more accurately referred to as pneumonitis.

Viral pneumonia is a pneumonia caused by a virus. Viruses are one of the two major causes of pneumonia, the other being bacteria; less common causes are fungi and parasites. Viruses are the most common cause of pneumonia in children, while in adults bacteria are a more common cause. Symptoms of viral pneumonia include fever, non-productive cough, runny nose, and systemic symptoms (e.g. myalgia, headache). Different viruses cause different symptoms.

### Cause

Common causes of viral pneumonia are:
Influenza virus A and B
Respiratory syncytial virus (RSV)
Human parainfluenza viruses (in children)

Rarer viruses that commonly result in pneumonia include:
Adenoviruses (in military recruits)
Metapneumovirus
Severe acute respiratory syndrome coronavirus (SARS-CoV)
Severe acute respiratory syndrome coronavirus 2 (SARS-CoV-2)
Middle East respiratory syndrome virus (MERS-CoV)
Hantaviruses

Viruses that primarily cause other diseases, but sometimes cause pneumonia include:
Herpes simplex virus (HSV), mainly in newborns or young children
Varicella-zoster virus (VZV)
Measles virus
Rubella virus
Cytomegalovirus (CMV), mainly in people with immune system problems
Smallpox virus
Dengue virus

The most commonly identified agents in children are respiratory syncytial virus, rhinovirus, human metapneumovirus, human bocavirus, and parainfluenza viruses.

### Pathophysiology

Viruses must invade cells in order to reproduce. Typically, a virus will reach the lungs by traveling in droplets through the mouth and nose with inhalation. There, the virus invades the cells lining the airways and the alveoli. This invasion often leads to cell death either through direct killing by the virus or by self-destruction through apoptosis.

Further damage to the lungs occurs when the immune system responds to the infection. White blood cells, in particular lymphocytes, are responsible for activating a variety of chemicals (cytokines) which cause leaking of fluid into the alveoli. The combination of cellular destruction and fluid-filled alveoli interrupts the transportation of oxygen into the bloodstream.

In addition to the effects on the lungs, many viruses affect other organs and can lead to illness affecting many different bodily functions. Some viruses also make the body more susceptible to bacterial infection; for this reason, bacterial pneumonia often complicates viral pneumonia.

### Treatment

In cases of viral pneumonia where influenza A or B are thought to be causative agents, patients who are seen within 48 hours of symptom onset may benefit from treatment with oseltamivir or zanamivir. Respiratory syncytial virus (RSV) has no direct acting treatments, but ribavirin is indicated for severe cases. Herpes simplex virus and varicella-zoster virus infections are usually treated with aciclovir, whilst ganciclovir is used to treat cytomegalovirus. There is no known efficacious treatment for pneumonia caused by SARS coronavirus, MERS coronavirus, adenovirus, hantavirus, or parainfluenza. Care is largely supportive.

Blood oxygen level is a measure of how much oxygen your red blood cells are carrying. Blood oxygen level can be measured with an arterial blood gas (ABG) test and/or a pulse oximeter. A measurement of blood oxygen level is called oxygen saturation level. The measurement may be referred to as SaO₂. Blood oxygen level may also be called PaO₂ when an ABG test is done or an O₂ sat (SpO₂) when it is measured with a pulse oximeter. A normal ABG level for healthy lungs falls between 80 and 100 millimeters of mercury (mm Hg). If a pulse oximeter is used, a normal reading may be between 95 and 100%. A below-normal blood oxygen level is hypoxemia. As used herein, hypoxemia includes an ABG level below 80 mm Hg (e.g., at or below 70 mm Hg, e.g., at or below 60 mm Hg, e.g., at or below 50 mm Hg, e.g., below 50 mm Hg) and/or an SpO₂ below 95% percent (e.g., below 90%).

The methods of the subject invention include administration of a therapeutically effective amount of a diffusion enhancing compound such as TSC.

The diffusion enhancing compound is a bipolar trans carotenoid salt having the formula:

YZ-TCRO-ZY,
where:
Y=a cation which can be the same or different,
Z=a polar group which can the same or different and which is associated with the cation,
TCRO=a linear trans carotenoid skeleton with conjugated carbon-carbon double bonds and single bonds, and having pendant groups X, wherein the pendant groups X, which can be the same or different, are a linear or branched hydrocarbon group having 10 or less carbon atoms, or a halogen.

Advantageously, the bipolar trans carotenoid salt is trans sodium crocetinate (TSC) (e.g., synthetic TSC), shown as Formula I below.

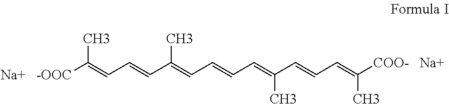

In one embodiment, the absorbency (e.g., in an aqueous solution) of the bipolar trans carotenoid salt (e.g., trans sodium crocetinate) at the highest peak which occurs in the visible wavelength range divided by the absorbency of a peak occurring in the ultraviolet wavelength range is greater than 7 (e.g., 7 to 8.5), e.g., greater than 7.5 (e.g., 7.5 to 9, e.g., 7.5 to 8.5), e.g., greater than 8 (e.g., 8 to 8.8), e.g., greater than 8.5. In another embodiment, the absorbency (e.g., in an aqueous solution) of the TSC at the highest peak which occurs in the visible wavelength range divided by the absorbency of a peak occurring in the ultraviolet wavelength range is greater than 7 (e.g., 7 to 8.5), e.g., greater than 7.5 (e.g., 7.5 to 9, e.g., 7.5 to 8.5), e.g., greater than 8 (e.g., 8 to 8.8), e.g., greater than 8.5.

The bipolar trans carotenoid salt (e.g., trans sodium crocetinate) is at least 90% pure as measured by high performance liquid chromatography (HPLC), e.g., ≥95% pure as measured by HPLC, e.g., ≥96% pure as measured by HPLC. In an advantageous embodiment, the TSC is at least 90% pure as measured by high performance liquid chromatography (HPLC), e.g., ≥95% pure as measured by HPLC, e.g., ≥96% pure as measured by HPLC.

In an advantageous embodiment, the bipolar trans carotenoid salt is in a composition also comprising a cyclodextrin. For instance, wherein TSC is in a composition also comprising a cyclodextrin (e.g., wherein the TSC is in a lyophilized composition with a cyclodextrin).

Advantageously, the cyclodextrin is gamma-cyclodextrin. For instance, the bipolar trans carotenoid salt is TSC which is in a composition also comprising gamma-cyclodextrin (e.g., wherein the TSC is in a lyophilized composition with gamma-cyclodextrin).

In an embodiment of the invention, the composition further comprises mannitol.

The diffusion enhancing compound is administered intravenously or intramuscularly (e.g., as an intravenous injection or infusion or intramuscular injection).

Advantageously, the diffusion enhancing compound is admixed with sterile water for injection to form an injection. TSC is administered intravenously or intramuscularly (e.g., as an intravenous injection or infusion or intramuscular injection). For instance, wherein TSC is admixed with sterile water for injection to form an injection.

Advantageously, the diffusion enhancing compound is TSC and is administered at a dose of 0.05-5 mg/kg, e.g., 0.05-2.5 mg/kg, e.g., 0.2-2 mg/kg, e.g., 0.15-0.35 mg/kg, e.g., 0.25 mg/kg. Specifically, TSC can be administered at a dose of 0.05-2.5 mg/kg, e.g., 0.2-2 mg/kg, e.g., 0.15-0.35 mg/kg, e.g., 0.25 mg/kg, three or more days per week, e.g., five or more days per week, e.g., each day of the week.

In another embodiment, provided is a diffusion enhancing compound (e.g., a bipolar trans carotenoid salt (e.g., TSC) for use in any of the methods described herein.

In another embodiment, provided is use of a diffusion enhancing compound (e.g., a bipolar trans carotenoid salt (e.g., TSC) in the manufacture of a medicament for any of the methods described herein.

In another embodiment, provided is a pharmaceutical composition comprising an effective amount of a diffusion enhancing compound (e.g., a bipolar trans carotenoid salt (e.g., TSC) for use in any of the methods described herein.

### Compounds and Compositions of the Invention

### Diffusion Enhancing Compounds

The diffusion enhancing compounds of the invention include those compounds described in U.S. Pat. Nos. 7,759,506, 8,030,350, 8,901,174 and 8,206,751, each of which is hereby incorporated by reference in its entirety.

Included are bipolar trans carotenoid compounds having the formula:

YZ-TCRO-ZY

where:
Y=a cation
Z=a polar group which is associated with the cation, and
TCRO=trans carotenoid skeleton,
such as TSC.

More specifically, the subject invention relates to trans carotenoids including trans carotenoid diesters, dialcohols, diketones and diacids, bipolar trans carotenoids (BTC), and bipolar trans carotenoid salts (BTCS) compounds and synthesis of such compounds having the structure:

YZ-TCRO-ZY

where:
Y (which can be the same or different at the two ends)=H or a cation other than H, preferably Na⁺ or K⁺ or Li⁺. Y is advantageously a monovalent metal ion. Y can also be an organic cation, e.g., R₄N⁺, R₃S⁺, where R is H, or CₙH₂ₙ₊₁ where n is 1-10, advantageously 1-6. For example, R can be methyl, ethyl, propyl or butyl.
Z (which can be the same or different at the two ends)=polar group which is associated with H or the cation. Optionally including the terminal carbon on the carotenoid (or carotenoid related compound), this group can be a carboxyl (COO⁻) group or a CO group (e.g. ester, aldehyde or ketone group), or a hydroxyl group. This group can also be a sulfate group (OSO₃⁻) or a monophosphate group (OPO₃⁻), (OP(OH)O₂⁻), a diphosphate group, triphosphate or combinations thereof. This group can also be an ester group of COOR where the R is CₙH₂ₙ₊₁.
TCRO=trans carotenoid or carotenoid related skeleton (advantageously less than 100 carbons) which is linear, has pendant groups (defined below), and typically comprises “conjugated” or alternating carbon-carbon double and single bonds (in one embodiment, the TCRO is not fully conjugated as in a lycopene). The pendant groups (X) are typically methyl groups but can be other groups as discussed below. In an advantageous embodiment, the units of the skeleton are joined in such a manner that their arrangement is reversed at the center of the molecule. The 4 single bonds that surround a carbon-carbon double bond all lie in the same plane. If the pendant groups are on the same side of the carbon-carbon double bond, the groups are designated as cis (also known as “Z”); if they are on the opposite side of the carbon-carbon bond, they are designated as trans (also known as “E”). Throughout this case, the isomers will be referred to as cis and trans.

The compounds of the subject invention are trans. The cis isomer typically is a detriment—and results in the diffusivity not being increased. In one embodiment, a cis isomer can be utilized where the skeleton remains linear. The placement of the pendant groups can be symmetric relative to the central point of the molecule or can be asymmetric so that the left side of the molecule does not look the same as the right side of the molecule either in terms of the type of pendant group or their spatial relationship with respect to the center carbon.

The pendant groups X (which can be the same or different) are hydrogen (H) atoms, or a linear or branched hydrocarbon group having 10 or less carbons, advantageously 4 or less, (optionally containing a halogen), or a halogen. X could also be an ester group (COO—) or an ethoxy/methoxy group. Examples of X are a methyl group (CH₃), an ethyl group (C₂H₅), a phenyl or single aromatic ring structure with or without pendant groups from the ring, a halogen-containing alkyl group (C1-C10) such as CH₂Cl, or a halogen such as Cl or Br or a methoxy (OCH₃) or ethoxy (OCH₂CH₃). The pendant groups can be the same or different but the pendant groups utilized must maintain the skeleton as linear.

Although many carotenoids exist in nature, carotenoid salts do not. Commonly-owned U.S. Pat. No. 6,060,511 hereby incorporated by reference in its entirety, relates to trans sodium crocetinate (TSC). The TSC was made by reacting naturally occurring saffron with sodium hydroxide followed by extractions that selected primarily for the trans isomer.

The presence of the cis and trans isomers of a carotenoid or carotenoid salt can be determined by looking at the ultraviolet-visible spectrum for the carotenoid sample dissolved in an aqueous solution. Given the spectrum, the value of the absorbence of the highest peak which occurs in the visible wave length range of 380 to 470 nm (the number depending on the solvent used and the chain length of the BTC or BTCS. The addition of pendant groups or differing chain lengths will change this peak absorbance but someone skilled in the art will recognize the existence of an absorbance peak in the visible range corresponding to the conjugated backbone structure of these molecules.) is divided by the absorbency of the peak which occurs in the UV wave length range of 220 to 300 nm can be used to determine the purity level of the trans isomer. When the trans carotenoid diester (TCD) or BTCS is dissolved in water, the highest visible wave length range peak will be at between 380 nm to 470 nm (depending on the exact chemical structure, backbone length and pendant groups) and the UV wave length range peak will be between 220 to 300 nm. According to M. Craw and C. Lambert, Photochemistry and Photobiology, Vol. 38 (2), 241-243 (1983) hereby incorporated by reference in its entirety, the result of the calculation (in that case crocetin was analyzed) was 3.1, which increased to 6.6 after purification.

Performing the Craw and Lambert analysis, using a cuvette designed for UV and visible wavelength ranges, on the trans sodium salt of crocetin of commonly owned U.S. Pat. No. 6,060,511 (TSC made by reacting naturally occurring saffron with sodium hydroxide followed by extractions which selected primarily for the trans isomer), the value obtained averages about 6.8. Performing that test on the synthetic TSC of the subject invention, that ratio is greater than 7.0 (e.g. 7.0 to 8.5), advantageously greater than 7.5 (e.g. 7.5-8.5), most advantageously greater than 8. The synthesized material is a “purer” or highly purified trans isomer.

### Formulation and Administration of the Compounds and Compositions of the Invention

A detailed description of formulation and administration of diffusing enhancing compounds can be found in commonly owned U.S. Pat. No. 8,293,804, U.S. application Ser. No. 12/801,726, and U.S. Pat. No. 8,206,751, each of which is hereby incorporated by reference in its entirety. A detailed description of formulation and administration of diffusing enhancing compounds can also be found in commonly owned U.S. Pat. No. 8,030,350, which is hereby incorporated by reference in its entirety.

A diffusion enhancing compound such as TSC can be administered by various routes for rapid delivery to the hypoxic tissue. For example, the compound, which can be formulated with other compounds including excipients, can be administered at the proper dosage as an intravenous injection (IV) or infusion, or an intramuscular injection (IM).

The IV injection route is an advantageous route for giving TSC for many of the uses of the subject application. Typically, a diffusion enhancing compound such as TSC is administered as soon as possible if a thrombus is believed present.

In addition to intravenous injection, routes of administration for specially formulated trans carotenoid molecules include intramuscular injection, delivery by inhalation, oral administration and transdermal administration.

### Cyclodextrins

In order to administer some pharmaceuticals, it is necessary to add another compound which will aid in increasing the absorption/solubility/concentration of the active pharmaceutical ingredient (API). Such compounds are called excipients, and cyclodextrins are examples of excipients. Cyclodextrins are cyclic carbohydrate chains derived from starch. They differ from one another by the number of glucopyranose units in their structure. The parent cyclodextrins contain six, seven and eight glucopyranose units, and are referred to as alpha, beta and gamma cyclodextrins respectively. Cyclodextrins were first discovered in 1891, and have been used as part of pharmaceutical preparations for several years.

Cyclodextrins are cyclic (alpha-1,4)-linked oligosaccharides of alpha-D-glucopyranose containing a relatively hydrophobic central cavity and hydrophilic outer surface. In the pharmaceutical industry, cyclodextrins have mainly been used as complexing agents to increase the aqueous solubility of poorly water-soluble drugs, and to increase their bioavailability and stability. In addition, cyclodextrins are used to reduce or prevent gastrointestinal or ocular irritation, reduce or eliminate unpleasant smells or tastes, prevent drug-drug or drug-additive interactions, or even to convert oils and liquid drugs into microcrystalline or amorphous powders.

Although the BTC compounds are soluble in water, the use of the cyclodextrins can increase that solubility even more so that a smaller volume of drug solution can be administered for a given dosage.

There are a number of cyclodextrins that can be used with the Compounds of the Invention. See for example, U.S. Pat. No. 4,727,064, hereby incorporated by reference in its entirety. Advantageous cyclodextrins are γ-cyclodextrin, 2-hydroxylpropyl-γ-cyclodextrin and 2-hydroxylpropyl-β-cyclodextrin, or other cyclodextrins which enhance the solubility of the BTC.

The use of gamma-cyclodextrin with TSC increases the solubility of TSC in water by 3-7 times. Although this is not as large a factor as seen in some other cases for increasing the solubility of an active agent with a cyclodextrin, it is important in allowing for the parenteral administration of TSC in smaller volume dosages to humans (or animals). Dosages of TSC and gamma-cyclodextrin have resulted in aqueous solutions containing as much as 44 milligrams of TSC per ml of solution, with an advantageous range of 20-30 mg/ml of solution. The solutions need not be equal-molar. The incorporation of the gamma cyclodextrin also allows for TSC to be absorbed into the blood stream when injected intramuscularly. Absorption is quick, and efficacious blood levels of TSC are reached quickly (as shown in rats).

The cyclodextrin formulation can be used with other trans carotenoids and carotenoid salts. The subject invention also includes novel compositions of carotenoids which are not salts (e.g. acid forms such as crocetin, crocin or the intermediate compounds noted above) and a cyclodextrin. In other words, trans carotenoids which are not salts can be formulated with a cyclodextrin. Mannitol can be added for osmolality, or the cyclodextrin BTC mixture can be added to isotonic saline (see below).

The amount of the cyclodextrin used is that amount which will contain the trans carotenoid but not so much that it will not release the trans carotenoid. Advantageously, the ratio of cyclodextrin to BTC, e.g., TSC, is 4 to 1 or 5 to 1. See also U.S. Pat. No. 8,974,822, the content of which is hereby incorporated by reference in its entirety.

### Cyclodextrin-Mannitol

A trans carotenoid such as TSC can be formulated with a cyclodextrin as noted above and a non-metabolized sugar such as mannitol (e.g. d-mannitol to adjust the osmotic pressure to be the same as that of blood). Solutions containing over 20 mg TSC/ml of solution can be made this way. This solution can be added to isotonic saline or to other isotonic solutions in order to dilute it and still maintain the proper osmolality.

### Mannitol/Acetic Acid

A BTCS such as TSC can be formulated with mannitol such as d-mannitol, and a mild buffering agent such as acetic acid or citric acid to adjust the pH. The pH of the solution should be around 8 to 8.5. It should be close to being an isotonic solution, and, as such, can be injected directly into the blood stream.

### Water+Saline

A BTCS such as TSC can be dissolved in water (advantageously injectable water). This solution can then be diluted with water, normal saline, Ringer's lactate or phosphate buffer, and the resulting mixture either infused or injected.

### Buffers

A buffer such as glycine, bicarbonate, or sodium carbonate can be added to the formulation at a level of about 50 mM for stability of the BCT such as TSC.

### TSC and Gamma-Cyclodextrin

The ratio of TSC to cyclodextrin is based on TSC:cyclodextrin solubility data. For example, 20 mg/ml TSC, 8% gamma cyclodextrin, 50 mM glycine, 2.33% mannitol with pH 8.2+/−0.5, or 10 mg/ml TSC and 4% cyclodextrin, or 5 mg/ml and 2% cyclodextrin. The ratios of these ingredients can be altered somewhat, as is obvious to one skilled in this art.

Mannitol can be used to adjust osmolality and its concentration varies depending on the concentration of other ingredients. The glycine is held constant. TSC is more stable at higher pHs. pH of around 8.2+/−0.5 is required for stability and physiological compatibility. The use of glycine is compatible with lyophilization. Alternatively, the TSC and cyclodextrin is formulated using a 50 mM bicarbonate buffer in place of the glycine.

### Endotoxin Removal of Gamma-Cyclodextrin

Commercially available pharmaceutical grade cyclodextrin has endotoxin levels that are incompatible with intravenous injection. The endotoxin levels must be reduced in order to use the cyclodextrin in a BTC formulation intended for intravenous injection.

### Kits and Dual Chamber Delivery Systems

The diffusion enhancing compound such as TSC can be lyophilized and put in a vial which can be part of a vial kit system which also includes a vial with diluent such as water for injection, and a syringe for administration.

Dual-chamber delivery systems allow reconstitution of the lyophilized diffusion enhancing compound directly inside the system be it a syringe or a cartridge. The lyophilized diffusion enhancing compound such as TSC is located in one chamber and the diluent (e.g. water for injection) in the other. The drug is reconstituted just before administration. It is a simple and controllable process completed in a few easy steps.

In one embodiment, the diffusion enhancing compound such as TSC is loaded in an auto-injector. An auto-injector (or auto-injector) is a medical device designed to deliver a dose of a particular drug. Most auto-injectors are spring-loaded syringes. By design, auto-injectors are easy to use and are intended for self-administration by patients, or administration by untrained personnel. The site of injection is typically the thigh or the buttocks. The auto-injector typically keeps the needle tip shielded prior to injection and also has a passive safety mechanism to prevent accidental firing (injection). Injection depth can be adjustable or fixed and a function for needle shield removal can be incorporated. Just by pressing a button, the syringe needle is automatically inserted and the drug is delivered.

### Uses of the Compounds and Compositions of the Invention

The subject invention provides methods for the treatment of human patients having, or diagnosed as having, a viral or bacterial induced respiratory disease with hypoxemia, with a diffusion enhancing compound of the invention. Included are methods of treating influenza, a corona virus infection including Covid-19, and bacterial or viral pneumonia.

The diffusion enhancing compound can be administered by various routes. For example, the compound (which can be formulated with other compounds), can be administered at the proper dosage as an intravenous injection or infusion, an intramuscular injection, or in an oral form. The IV injection route is an advantageous route for giving a diffusion enhancing compound such as TSC. The patient can be given a diffusion enhancing compound such as TSC, e.g., by IV injection or infusion, IM, or orally, or, e.g., 1-2 hours prior to a procedure (e.g., prior to administration of supplemental oxygen, for example, by nasal cannula, noninvasive ventilation (e.g., via mask), mechanical ventilation (e.g., endotracheal intubation or tracheostomy and mechanical ventilation), and/or extracorporeal membrane oxygenation)), at a dosage in the range of 0.05-5 mg/kg, e.g., 0.05-2.5 mg/kg or 0.1-2 mg/kg.

The diffusion enhancing compound (e.g., bipolar trans carotenoid salt, e.g., TSC) may be administered up to four times a day, e.g., up to four times a day for up to 15 days, e.g., four times a day for 5 days. The diffusion enhancing compound (e.g., bipolar trans carotenoid salt, e.g., TSC) may be administered every 6 hours, e.g., every 6 hours for up to 15 days, e.g., every 6 hours for 5 days.

In certain embodiments, the diffusion enhancing compound, e.g. TSC, is administered in conjunction with the patient receiving supplemental oxygen or other compounds used in the treatment of viral or bacterial induced respiratory disease such as antivirals and/or antibiotics.

Provided is a method (Method 1) of treating a patient (e.g., a human) in need thereof having a viral or bacterial induced respiratory disease with hypoxemia comprising administering a diffusion enhancing compound to said patient. Provided is a method (Method 1) of treating a viral or bacterial induced respiratory disease with hypoxemia in a patient in need thereof (e.g., a human) comprising administering a diffusion enhancing compound to said patient.

Further provided is a method (Method 2) of prophylaxis and/or treatment of hypoxemia consequent to a viral or bacterial induced respiratory disease in a patient (e.g., a human) in need thereof comprising administering a diffusion enhancing compound to said patient.

Further provided is a method (Method 3) of prophylaxis and/or treatment of acute respiratory distress syndrome associated with a viral or bacterial respiratory disease in a patient (e.g., a human) in need thereof comprising administering a diffusion enhancing compound to said patient.

Further provided is a method (Method 4) of prophylaxis and/or treatment of acute respiratory distress syndrome consequent to a viral or bacterial respiratory disease in a patient (e.g., a human) in need thereof comprising administering a diffusion enhancing compound to said patient. For instance, provided is a method of prophylaxis and/or treatment of mild acute respiratory distress syndrome consequent to a viral or bacterial respiratory disease in a patient (e.g., a human) in need thereof comprising administering a diffusion enhancing compound to said patient. For instance, provided is a method of prophylaxis and/or treatment of moderate acute respiratory distress syndrome consequent to a viral or bacterial respiratory disease in a patient (e.g., a human) in need thereof comprising administering a diffusion enhancing compound to said patient. For instance, provided is a method of prophylaxis and/or treatment of severe acute respiratory distress syndrome consequent to a viral or bacterial respiratory disease in a patient (e.g., a human) in need thereof comprising administering a diffusion enhancing compound to said patient.

Further provided is a method (Method 5) of prophylaxis and/or treatment of multiple organ failure consequent to a viral or bacterial respiratory disease in a patient (e.g., a human) in need thereof comprising administering a diffusion enhancing compound to said patient.

Further provided is a method (Method 6) of mitigation, control, and/or treatment of one or more of hypoxemia, acute respiratory distress syndrome, and multiple organ failure consequent to a viral or bacterial respiratory disease in a patient (e.g., a human) in need thereof comprising administering a diffusion enhancing compound to said patient. For instance, provided are methods of mitigation, control, and/or treatment of one or more of hypoxemia, acute respiratory distress syndrome, and multiple organ failure consequent to a viral or bacterial respiratory disease in a patient (e.g., a human) in need thereof comprising administering a diffusion enhancing compound to said patient, wherein said patient has mild acute respiratory distress syndrome, moderate acute respiratory distress syndrome, or severe acute respiratory distress syndrome.

Further provided is a method (Method 7) of improving blood oxygenation in a patient in need thereof (e.g., a human with a viral or bacterial respiratory disease, e.g. a human with hypoxemia consequent to a viral or bacterial respiratory disease) comprising administering a diffusion enhancing compound to said patient.

Further provided is a method (Method 8) of treating a patient (e.g., a human) in need thereof having a viral or bacterial induced respiratory disease comprising administering a diffusion enhancing compound to said patient. Provided is a method (Method 8) of treating a viral or bacterial induced respiratory disease in a patient in need thereof (e.g., a human) comprising administering a diffusion enhancing compound to said patient. For instance, a method of treating a viral or bacterial induced respiratory disease in a patient in need thereof (e.g., a human) comprising administering a diffusion enhancing compound to said patient, wherein said patient has no respiratory disease symptoms (e.g., no hypoxemia).

Further provided are any one of Methods 1-8 as follows:
1.1. Any one of Methods 1-8, wherein said diffusion enhancing compound is a bipolar trans carotenoid salt having the formula:

YZ-TCRO-ZY,
where:
Y=a cation which can be the same or different,
Z=a polar group which can the same or different and which is associated with the cation,
TCRO=a linear trans carotenoid skeleton with conjugated carbon-carbon double bonds and single bonds, and having pendant groups X, wherein the pendant groups X, which can be the same or different, are a linear or branched hydrocarbon group having 10 or less carbon atoms, or a halogen.
1.2. Any one of Methods 1-8 or 1.1, wherein the bipolar trans carotenoid salt is trans sodium crocetinate (TSC) (e.g., synthetic TSC).
1.3. Any one of Methods 1-8, 1.1, or 1.2, wherein the absorbency (e.g., in an aqueous solution) of the bipolar trans carotenoid salt (e.g., trans sodium crocetinate) at the highest peak which occurs in the visible wavelength range divided by the absorbency of a peak occurring in the ultraviolet wavelength range is equal to or greater than 7 (e.g., 7 to 8.5), e.g., equal to or greater than 7.5 (e.g., 7.5 to 9, e.g., 7.5 to 8.5), e.g., equal to or greater than 8 (e.g., 8 to 8.8), e.g., greater than 8.5. For instance, wherein the absorbency (e.g., in aqueous solution) of the highest peak which occurs in the visible wavelength range of 380 to 470 nm divided by the absorbency of the peak which occurs in the UV wavelength range of 220 to 300 nm is equal to or greater than 7 (e.g., 7 to 8.5), e.g., equal to or greater than 7.5 (e.g., 7.5 to 9, e.g., 7.5 to 8.5), e.g., equal to or greater than 8 (e.g., 8 to 8.8), e.g., greater than 8.5.
1.4. Any one of Methods 1-8 or 1.1-1.3, wherein the absorbency (e.g., in an aqueous solution) of the TSC at the highest peak which occurs in the visible wavelength range divided by the absorbency of a peak occurring in the ultraviolet wavelength range is equal to or greater than 7 (e.g., 7 to 8.5), e.g., equal to or greater than 7.5 (e.g., 7.5 to 9, e.g., 7.5 to 8.5), e.g., equal to or greater than 8 (e.g., 8 to 8.8), e.g., greater than 8.5. For instance, wherein the absorbency (e.g., in aqueous solution) of the TSC at the highest peak which occurs in the visible wavelength range of 380 to 470 nm divided by the absorbency of the peak which occurs in the UV wavelength range of 220 to 300 nm is equal to or greater than 7 (e.g., 7 to 8.5), e.g., equal to or greater than 7.5 (e.g., 7.5 to 9, e.g., 7.5 to 8.5), e.g., equal to or greater than 8 (e.g., 8 to 8.8), e.g., greater than 8.5.
1.5. Any one of Methods 1-8 or 1.1-1.4, wherein the bipolar trans carotenoid salt (e.g., trans sodium crocetinate) is at least 90% pure as measured by high performance liquid chromatography (HPLC), e.g., ≥95% pure as measured by HPLC, e.g., ≥96% pure as measured by HPLC.
1.6. Any one of Methods 1-8 or 1.1-1.5, wherein the TSC is at least 90% pure as measured by high performance liquid chromatography (HPLC), e.g., ≥95% pure as measured by HPLC, e.g., ≥96% pure as measured by HPLC.
1.7. Any one of Methods 1-8 or 1.1-1.6, wherein the bipolar trans carotenoid salt is in a composition also comprising a cyclodextrin. For instance, wherein TSC is in a composition also comprising a cyclodextrin (e.g., wherein the TSC is in a lyophilized composition with a cyclodextrin).
1.8. Method 1.7, wherein the cyclodextrin is selected from the group consisting of alpha cyclodextrin, beta cyclodextrin, 2-hydroxypropyl-beta-cyclodextrin, 2-hydroxypropyl-gamma-cyclodextrin, and gamma cyclodextrin.
1.9. Method 1.8, wherein the cyclodextrin is selected from the group consisting of alpha cyclodextrin, 2-hydroxypropyl-beta-cyclodextrin, and gamma cyclodextrin.
1.10. Method 1.9, wherein the cyclodextrin is gamma-cyclodextrin. For instance, wherein the bipolar trans carotenoid salt is TSC which is in a composition also comprising gamma-cyclodextrin (e.g., wherein the TSC is in a lyophilized composition with gamma-cyclodextrin).
1.11. Any one of Methods 1-8 or 1.1-1.10, wherein the composition further comprises mannitol.
1.12. Any one of Methods 1-8 or 1.1-1.11, wherein the diffusion enhancing compound is administered intravenously or intramuscularly (e.g., as an intravenous bolus injection or intravenous infusion or intramuscular injection). For instance, any one of Methods 1-8 or 1.1-1.11, wherein the diffusion enhancing compound is admixed with sterile water for injection to form an injection. Any one of Methods 1-8 or 1.1-1.11, wherein TSC is administered intravenously or intramuscularly (e.g., as an intravenous bolus injection or intravenous infusion or intramuscular injection). For instance, any one of Methods 1-8 or 1.1-1.11, wherein TSC is admixed with sterile water for injection to form an injection.
1.13. Any one of Methods 1-8 or 1.1-1.12, wherein the diffusion enhancing compound is administered intravenously (e.g., as an intravenous bolus injection).
1.14. Any one of Methods 1-8 or 1.1-1.13, wherein the diffusion enhancing compound is administered at a dose of 0.05-5 mg/kg, e.g., 0.05-2.5 mg/kg or 0.1-2 mg/kg. For instance, any one of Methods 1-8 or 1.1-1.13, wherein the diffusion enhancing compound is TSC and is administered at a dose of 0.05-5 mg/kg, e.g., 0.05-2.5 mg/kg, e.g., 0.2-2 mg/kg, e.g., 0.1-2 mg/kg, e.g., 0.25 mg/kg or 0.5 mg/kg or 1 mg/kg or 1.5 mg/kg or 2 mg/kg or 2.5 mg/kg.
1.15. Any one of Methods 1-8 or 1.1-1.14, wherein the diffusion enhancing compound is TSC and is administered at a dose of 0.25-1.5 mg/kg, e.g., 0.25 mg/kg or 0.5 mg/kg or 1 mg/kg or 1.5 mg/kg. Or any one of Methods 1-8 or 1.1-1.14, wherein the diffusion enhancing compound is TSC and is administered at a dose of 2 mg/kg or 2.5 mg/kg.
1.16. Any one of Methods 1-8 or 1.1-1.15, wherein the diffusion enhancing compound is TSC and is administered at a dose of 0.25-1.5 mg/kg three times per week.
1.17. Any one of Methods 1-8 or 1.1-1.16, wherein the diffusion enhancing compound is TSC and is administered at a dose of 0.25 mg/kg three times per week.
1.18. Any one of Methods 1-8 or 1.1-1.17, wherein the diffusion enhancing compound is TSC and is administered at a dose of 0.25 mg/kg 3 days per week, for 3 weeks.
1.19. Any one of Methods 1-8 or 1.1-1.18, wherein the diffusion enhancing compound is TSC and is administered 4 times per day.
1.20. Any one of Methods 1-8 or 1.1-1.19, wherein the diffusion enhancing compound is TSC and is administered every 6 hours.
1.21. Any one of Methods 1-8 or 1.1-1.20, wherein the diffusion enhancing compound is TSC and is administered for 15 days or less, e.g., for 14 days or less, e.g., for 5 days or less, e.g., for 5 days. For instance, any one of Methods 1-8 or 1.1-1.20, wherein the diffusion enhancing compound is TSC and is administered for up to 15 days, e.g., for up to 5 days.
1.22. Any one of Methods 1-8, 1.1-1.15, or 1.19-1.21, wherein the diffusion enhancing compound is TSC and is administered each day for 3 weeks or less, e.g., for 15 days or less, e.g., for 14 days or less, e.g., for 5 days or less, e.g., for 5 days. For instance, any one of Methods 1-8, 1.1-1.15, or 1.19-1.21, wherein the diffusion enhancing compound is TSC and is administered 4 times per day (e.g., every 6 hours) each day for 3 weeks or less, e.g., for 15 days or less, e.g., for 14 days or less, e.g., for 5 days or less, e.g., for 5 days. For instance, any one of Methods 1-8, 1.1-1.15, or 1.19-1.21, wherein the diffusion enhancing compound is TSC and is administered 4 times per day every 6 hours each day for 3 weeks or less, e.g., for 15 days or less, e.g., for 14 days or less, e.g., for 5 days or less, e.g., for 5 days.
1.23. Any one of Methods 1-8 or 1.1-1.22, wherein the disease is influenza.
1.24. Any one of Methods 1-8 or 1.1-1.22, wherein the disease is a corona virus infection.
1.25. Any one of Methods 1-8, 1.1-1.22, or 1.24, wherein the disease is COVID-19.
1.26. Method 1.25, wherein the disease is mild COVID-19 or moderate COVID-19.
1.27. Method 1.25, wherein the disease is severe COVID-19.
1.28. Any one of Methods 1-8 or 1.1-1.22, wherein the disease is bacterial or viral pneumonia.
1.29. Any one of Methods 1-8 or 1.1-1.28, wherein the patient is human (e.g., an adult, e.g., ≥18 years of age, e.g., ≥18 years of age and <60 years of age or ≥60 years of age).
1.30. Any one of Methods 1-8 or 1.1-1.29, wherein the patient has an SpO₂≤94% (e.g., as measured with a pulse oximeter) when breathing room air. For instance, any one of Methods 1-8 or 1.1-1.29, wherein the patient has an SpO₂<94% (e.g., as measured with a pulse oximeter) when breathing room air.
1.31. Any one of Methods 1-8 or 1.1-1.30, wherein the patient requires supplemental oxygen. Any one of Methods 1-8 or 1.1-1.30, wherein the diffusion enhancing compound (e.g., bipolar trans carotenoid salt, e.g., TSC) is administered to the patient shortly before the patient receives supplemental oxygen (for example, by nasal cannula, noninvasive ventilation (e.g., via mask), mechanical ventilation (e.g., endotracheal intubation or tracheostomy and mechanical ventilation), and/or extracorporeal membrane oxygenation)).
1.32. Any one of Methods 1-8 or 1.1-1.31, wherein the patient is receiving supplemental oxygen.
1.33. Any one of Methods 7 or 1.1-1.32, wherein the improvement by the diffusion enhancing compound (e.g., TSC) in blood oxygenation is measured by one or both of the SpO₂:FiO₂ (S:F) ratio (e.g., as measured by continuous pulse oximetry) and PaO₂/FiO₂ (P:F) ratio.
1.34. Any one of Methods 1-8 or 1.1-1.33, wherein the patient has mild acute respiratory distress syndrome or is at risk of developing mild acute respiratory distress syndrome. Or any one of Methods 1-8 or 1.1-1.33, wherein the patient is a patient diagnosed with a viral or bacterial respiratory disease and at risk of developing one or more of acute respiratory distress syndrome (e.g., mild, moderate, or severe), hypoxemia, and multiple organ failure.
1.35. Any one of Methods 1-8 or 1.1-1.33, wherein the patient has moderate acute respiratory distress syndrome or is at risk of developing moderate acute respiratory distress syndrome.
1.36. Any one of Methods 1-8 or 1.1-1.33, wherein the patient has severe acute respiratory distress syndrome or is at risk of developing severe acute respiratory distress syndrome.
1.37. Any one of Methods 1-8 or 1.1-1.36, wherein the patient has a World Heath Organization ordinal scale score of 3, 4, or 5.
1.38. Any one of Methods 1-8 or 1.1-1.37, wherein the patient is hospitalized but is not being given oxygen therapy (e.g., World Heath Organization ordinal scale score of 3).
1.39. Any one of Methods 1-8 or 1.1-1.37, wherein the patient is hospitalized and is being given oxygen (e.g., by mask or nasal prongs) (e.g., World Heath Organization ordinal scale score of 4).
1.40. Any one of Methods 1-8 or 1.1-1.37, wherein the patient is hospitalized and is being given non-invasive ventilation or high-flow oxygen (e.g., World Heath Organization ordinal scale score of 5).
1.41. Any one of Methods 1-8 or 1.1-1.36, wherein the patient is intubated and on mechanical ventilation.
1.42. Any one of Methods 1-8, 1.1-1.36, or 1.41, wherein the patient is intubated and on mechanical ventilation and is receiving additional organ support (e.g., is receiving one or more of a vasopressor, a renal replacement therapy, and extracorporeal membrane oxygenation (ECMO)).
1.43. Any one of Methods 1-8 or 1.1-1.42, wherein the patient achieves at least 1-point improvement from the patient's baseline on the WHO ordinal scale score from treatment. For instance, wherein the patient achieves at least 1-point improvement from the patient's baseline on the WHO ordinal scale score from treatment and wherein the improvement is maintained for at least 24 hours, e.g., at least 48 hours, e.g., at least 1 week, e.g., at least 10 days, e.g., at least 13 days, e.g., at least 2 weeks, e.g., wherein the improvement is maintained through Day 28, wherein Day 1 is the first day of treatment.
1.44. Any one of Methods 1-8 or 1.1-1.43, wherein the patient achieves a 0, 1, 2, or 3 (e.g., a 1, 2, or 3) on the WHO ordinal scale score from treatment. For instance, wherein the patient achieves a 0, 1, 2, or 3 (e.g., a 1, 2, or 3) on the WHO ordinal scale score from treatment and wherein the WHO ordinal scale score of 0, 1, 2, or 3 (e.g., 1, 2, or 3) is maintained for at least 24 hours, e.g., at least 48 hours, e.g., at least 1 week, e.g., at least 10 days, e.g., at least 13 days, e.g., at least 2 weeks, e.g., wherein the WHO ordinal scale score of 0, 1, 2, or 3 (e.g., 1, 2, or 3) is maintained through Day 28, wherein Day 1 is the first day of treatment.
1.45. Any one of Methods 1-8 or 1.1-1.44, wherein the patient does not progress to a WHO ordinal scale score of 6 or above.
1.46. Any one of Methods 1-8 or 1.1-1.45, wherein the method further comprises administering one or more of an antiviral (e.g., remdesivir and/or lopinavir/ritonavir), a corticosteroid (e.g., dexamethasone), and an immunomodulator (e.g., hydroxychloroquine) to the patient. Any one of Methods 1-8 or 1.1-1.45, wherein the method further comprises administering one or more of an antiviral (e.g., remdesivir and/or lopinavir/ritonavir), a corticosteroid (e.g., dexamethasone), an immunomodulator (e.g., hydroxychloroquine), and a monoclonal antibody (e.g., a B-cell maturation antigen (BCMA)-directed antibody, e.g., belantamab mafodotin-blmf) to the patient.
1.47. Any one of Methods 1-8 or 1.1-1.46, wherein the method further comprises administering remdesivir to the patient.
1.48. Any one of Methods 1-8 or 1.1-1.47, wherein the method further comprises administering dexamethasone to the patient.
1.49. Any one of Methods 1-8 or 1.1-1.48, wherein the method further comprises administering hydroxychloroquine, in free or pharmaceutically acceptable salt form (e.g., hydroxychloroquine sulfate), to the patient.
1.50. Any one of Methods 1-8 or 1.1-1.49, wherein the method results in an improvement in median time to recovery (e.g., shorter median time to recovery) compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound).
1.51. Any one of Methods 1-8 or 1.1-1.50, wherein the method results in an improvement in median time to recovery to 7 days (e.g., from 12 days to 7 days) compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound).
1.52. Any one of Methods 1-8 or 1.1-1.51, wherein the patient achieves (e.g., recovery is) a WHO ordinal scale score of 0, 1, 2, or 3 (e.g., 1, 2, or 3). For instance, wherein the patient achieves (e.g., recovery is) a WHO ordinal scale score of 0, 1, 2, or 3 (e.g., 1, 2, or 3) and wherein the WHO ordinal scale score of 0, 1, 2, or 3 (e.g., 1, 2, or 3) is maintained for at least 24 hours, e.g., at least 48 hours, e.g., at least 1 week, e.g., at least 10 days, e.g., at least 13 days, e.g., at least 2 weeks, e.g., wherein the WHO ordinal scale score of 0, 1, 2, or 3 (e.g., 1, 2, or 3) is maintained through Day 28, wherein Day 1 is the first day of treatment.
1.53. Any one of Methods 1-8 or 1.1-1.52, wherein the patient achieves (e.g., recovery is) a WHO ordinal scale score of 0, 1, 2, or 3 (e.g., 1, 2, or 3) and an improvement of at least 1 point on the WHO ordinal scale from the patient's baseline (e.g., wherein the improvement of at least 1 point is maintained for at least 24 hours, e.g., at least 48 hours, e.g., at least 1 week, e.g., at least 10 days, e.g., at least 13 days, e.g., at least 2 weeks, e.g., wherein the improvement of at least 1 point is maintained through Day 28, wherein Day 1 is the first day of treatment). For instance, wherein the patient achieves (e.g., recovery is) a WHO ordinal scale score of 0, 1, 2, or 3 (e.g., 1, 2, or 3) and an improvement of at least 1 point on the WHO ordinal scale from the patient's baseline (e.g., wherein the WHO ordinal score of 0, 1, 2, or 3 (e.g., 1, 2, or 3) and the improvement of at least 1 point is maintained for at least 24 hours, e.g., at least 48 hours, e.g., at least 1 week, e.g., at least 10 days, e.g., at least 13 days, e.g., at least 2 weeks, e.g., wherein the WHO ordinal scale score of 0, 1, 2, or 3 (e.g., 1, 2, or 3) and the improvement of at least 1 point is maintained through Day 28, wherein Day 1 is the first day of treatment). For instance, wherein the patient achieves (e.g., recovery is) a WHO ordinal scale score of 0, 1, 2, or 3 (e.g., 1, 2, or 3) and an improvement of at least 1 point on the WHO ordinal scale from the patient's baseline (e.g., wherein the WHO ordinal score of 0, 1, 2, or 3 (e.g., 1, 2, or 3) and the WHO ordinal scale score of 0, 1, 2, or 3 and the improvement of at least 1 point is maintained for at least 24 hours, e.g., at least 48 hours, e.g., at least 1 week, e.g., at least 10 days, e.g., at least 13 days, e.g., at least 2 weeks, e.g., wherein the WHO ordinal scale score of 0, 1, 2, or 3 (e.g., 1, 2, or 3) and the improvement of at least 1 point is maintained through Day 28, wherein Day 1 is the first day of treatment). For instance, a patient with a baseline WHO ordinal scale score of 3 that improves to a score of 0, 1, or 2 (e.g., 1 or 2) and the score of 0, 1, or 2 (e.g., 1 or 2) is maintained for at least 24 hours (e.g., at least 48 hours, e.g., at least 1 week, e.g., at least 10 days, e.g., at least 13 days, e.g., at least 2 weeks, e.g., maintained to Day 29, wherein Day 1 is the first day of treatment) has recovered. Also, for instance, a patient with a baseline WHO ordinal scale score of 4 or 5 that improves to a score of 0, 1, 2, or 3 (e.g., 1, 2, or 3) and the score of 0, 1, 2, or 3 (e.g., 1, 2, or 3) is maintained for at least 24 hours (e.g., at least 48 hours, e.g., at least 1 week, e.g., at least 10 days, e.g., at least 13 days, e.g., at least 2 weeks, e.g., maintained to Day 29, wherein Day 1 is the first day of treatment) has recovered. Day 29 evaluation of the WHO ordinal scale score may be the worst score from the previous day (Day 28).
1.54. Any one of Methods 1-8 or 1.1-1.53, wherein the patient's WHO ordinal scale score does not increase from baseline. For instance, wherein the patient's WHO ordinal scale score does not increase to 5, 6, or 7 (e.g., to 6 or 7).
1.55. Any one of Methods 1-8 or 1.1-1.54, wherein trans sodium crocetinate (TSC) is administered in a composition reconstituted with sterile water for injection comprising 20 mg/ml TSC, 8% w/v gamma cyclodextrin, 50 mM glycine, and 2.3% w/v mannitol.
1.56. Method 1.55, wherein the composition has a pH of 8.0-8.2.
1.57. Any one of Methods 1-8 or 1.1-1.56, wherein the patient has silent or apathetic hypoxemia (e.g., the patient has observable hypoxemia by an oxygen saturation (e.g., SpO₂) measurement, but shows minimal outward signs of respiratory distress).
1.58. Any one of Methods 1-8 or 1.1-1.57, wherein the method results in a reduction in the length of hospital stay compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound). For instance, any one of Methods 1-8 or 1.1-1.57, wherein the patient's time to discharge from a hospital is less compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound).
1.59. Any one of Methods 1-8 or 1.1-1.58, wherein the patient's time to a National Early Warning Score (NEWS) of ≤2 that is maintained for at least 24 hours is shorter compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound).
1.60. Any one of Methods 1-8 or 1.1-1.59, wherein the patient achieves one or more endpoints selected from primary and secondary endpoints described in Example 1 below.
1.61. Any one of Methods 1-8 or 1.1-1.60, wherein the patient has one or more of hypertension, diabetes, and an immune-related disease.
1.62. Any one of Methods 1-8 or 1.1-1.61, wherein (in the below, patients with the same baseline WHO ordinal scale score and/or with the same chronic diseases and/or taking the same chronic medications/therapies and/or with the same diagnosis may optionally be compared):
   the patient achieves a lower WHO ordinal scale score in a shorter time compared to a patient with the same baseline WHO ordinal scale score not administered the diffusion enhancing compound;
   the patient requires less oxygen therapy (e.g., oxygen delivered by nasal cannula, noninvasive ventilation (e.g., via mask), mechanical ventilation (e.g., endotracheal intubation or tracheostomy and mechanical ventilation), and/or extracorporeal membrane oxygenation) after administration of the diffusion enhancing compound compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound), for instance, the patient returns to breathing room air or to their baseline oxygen requirement (e.g., the patient's oxygen requirement pre-viral or pre-bacterial induced respiratory disease) in a shorter time (e.g., compared to a patient with the same baseline WHO ordinal scale score not administered the diffusion enhancing compound and/or compared to a patient with the same diagnosis not administered the diffusion enhancing compound), or, for instance, the patient does not require oxygen therapy (e.g., compared to a patient with the same baseline WHO ordinal scale score not administered the diffusion enhancing compound and/or compared to a patient with the same diagnosis not administered the diffusion enhancing compound);
   the patient's duration of (e.g., days on) oxygen therapy (e.g., oxygen delivered by nasal cannula, noninvasive ventilation (e.g., via mask), mechanical ventilation (e.g., endotracheal intubation or tracheostomy and mechanical ventilation), and/or extracorporeal membrane oxygenation) after administration of the diffusion enhancing compound is less compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound);
   the patient's duration of (e.g., days on) extracorporeal membrane oxygenation (ECMO) after administration of the diffusion enhancing compound is less compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound);
   the patient has improved blood oxygenation (e.g., as recorded by continuous pulse oximetry (SpO₂:FiO₂ ratio) and/or PaO₂/FiO₂ ratio) after administration of the diffusion enhancing compound compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound);
   the incidence of oxygen therapy (e.g., oxygen delivered by nasal cannula, noninvasive ventilation (e.g., via mask), mechanical ventilation (e.g., endotracheal intubation or tracheostomy and mechanical ventilation), and/or extracorporeal membrane oxygenation) for the patient after administration of the diffusion enhancing compound is less compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound);
   the patient requires less vasopressor (e.g., has more vasopressor free days (including no vasopressor administration)) after administration of the diffusion enhancing compound compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound), for instance, the patient returns to no vasopressor or to their baseline vasopressor requirement (e.g., the patient's vasopressor requirement pre-viral or pre-bacterial induced respiratory disease);
   the patient does not develop acute kidney injury (e.g., as defined by AKIN (Acute Kidney Injury Network) criteria);
   the patient requires less renal replacement therapy (RRT) (e.g., has more renal replacement therapy free days (including no renal replacement therapy administration)) or has no new renal replacement therapy for patients that are on chronic renal replacement therapy (e.g., chronic hemodialysis) after administration of the diffusion enhancing compound compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound), for instance, the patient returns to no renal replacement therapy or to their baseline renal replacement therapy requirement (e.g., the patient's renal replacement therapy requirement pre-viral or pre-bacterial induced respiratory disease);
   the patient does not have organ failure;
   the method results in a reduction in the length of stay in an intensive care unit for the patient after administration of the diffusion enhancing compound compared to a patient not administered the diffusion enhancing compound (e.g., compared to a patient with the same baseline WHO ordinal scale score not administered the diffusion enhancing compound and/or compared to a patient with the same diagnosis not administered the diffusion enhancing compound); and/or
   the patient does not develop respiratory failure (e.g., the patient does not have mechanical ventilation (e.g., endotracheal intubation or tracheostomy and mechanical ventilation), extracorporeal membrane oxygenation, oxygen delivered by nasal cannula, and/or noninvasive ventilation (e.g., via mask)).
1.63. Any one of Methods 1-8 or 1.1-1.62, wherein the diffusion enhancing compound is TSC and is administered at a dose of 0.25-5 mg/kg, e.g., 0.25 mg/kg or 0.5 mg/kg or 1 mg/kg or 1.5 mg/kg or 2 mg/kg or 2.5 mg/kg.
1.64. Any one of Methods 1-8 or 1.1-1.63, wherein the diffusion enhancing compound is TSC and is administered at a dose of 0.25-5 mg/kg, e.g., up to 0.25 mg/kg or up to 0.5 mg/kg or up to 1 mg/kg or up to 1.5 mg/kg or up to 2 mg/kg or up to 2.5 mg/kg or up to 5 mg/kg.
1.65. Any one of Methods 1-8 or 1.1-1.64, wherein the diffusion enhancing compound is TSC and is administered at a dose of 0.25-5 mg/kg, e.g., 0.25 mg/kg or 0.5 mg/kg or 1 mg/kg or 1.5 mg/kg or 2 mg/kg or 2.5 mg/kg, up to four times a day (e.g., four times a day). For instance, any one of Methods 1-8 or 1.1-1.64, wherein the diffusion enhancing compound is TSC and is administered at a dose of 0.25-5 mg/kg, e.g., 0.25 mg/kg or 0.5 mg/kg or 1 mg/kg or 1.5 mg/kg or 2 mg/kg or 2.5 mg/kg, up to four times a day (e.g., four times a day) for up to 15 days (e.g., for up to 5 days).
1.66. Any one of Methods 1-8 or 1.1-1.65, wherein the diffusion enhancing compound is TSC and is administered at a dose of 0.25-5 mg/kg, e.g., 0.25 mg/kg or 0.5 mg/kg or 1 mg/kg or 1.5 mg/kg or 2 mg/kg or 2.5 mg/kg, every 6 hours. For instance, any one of Methods 1-8 or 1.1-1.65, wherein the diffusion enhancing compound is TSC and is administered at a dose of 0.25-5 mg/kg, e.g., 0.25 mg/kg or 0.5 mg/kg or 1 mg/kg or 1.5 mg/kg or 2 mg/kg or 2.5 mg/kg, every 6 hours for up to 15 days (e.g., for up to 5 days).
1.67. Any one of Methods 1-8 or 1.1-1.66, wherein the diffusion enhancing compound is TSC and is administered multiple times a day (e.g., four times a day).
1.68. Any one of Methods 1-8 or 1.1-1.67, wherein the diffusion enhancing compound is TSC and is administered every 2 hours or every 3 hours or every 4 hours or every 5 hours or every 6 hours. For instance, any one of Methods 1-8 or 1.1-1.67, wherein the diffusion enhancing compound is TSC and is administered every 2 hours or every 3 hours or every 4 hours or every 5 hours or every 6 hours for up to 15 days (e.g., for up to 5 days).
1.69. Any one of methods 1-8 or 1.1-1.67, wherein the diffusion enhancing compound is TSC and is administered by a continuous intravenous infusion.
1.70. Any one of Methods 1-8 or 1.1-1.69, wherein the total dose (e.g., total daily dose) of the TSC does not result in a visual disturbance (e.g., a yellow visual disturbance).

Further provided is a kit comprising:
a) a container comprising a diffusion enhancing compound, and
b) instructions for using the diffusion enhancing compound to treat a patient having or suspected of having, a viral or bacterial induced respiratory disease by administering the diffusion enhancing compound at a dose of 0.05-5 mg/kg, e.g., 0.05-2.5 mg/kg.

Further provided is a kit comprising:
a) a container comprising a diffusion enhancing compound, and
b) instructions for using the diffusion enhancing compound to treat a patient according to any one of Methods 1-8 or 1.1-1.70.

Further provided is a diffusion enhancing compound (e.g., a bipolar trans carotenoid salt (e.g., TSC)), e.g., as described in any one of Methods 1-8 or 1.1-1.70, for use in any one of Methods 1-8 or 1.1-1.70.

Further provided is use of a diffusion enhancing compound (e.g., a bipolar trans carotenoid salt (e.g., TSC)), e.g., as described in any one of Methods 1-8 or 1.1-1.70, in the manufacture of a medicament for any one of Methods 1-8 or 1.1-1.70.

Further provided is a pharmaceutical composition comprising an effective amount of a diffusion enhancing compound (e.g., a bipolar trans carotenoid salt (e.g., TSC)), e.g., as described in any one of Methods 1-8 or 1.1-1.70, for use in any one of Methods 1-8 or 1.1-1.70.

In the methods disclosed herein (e.g., any one of Methods 1-8 or 1.1-1.70), the World Heath Organization ordinal scale score is as described in Example 1 below.

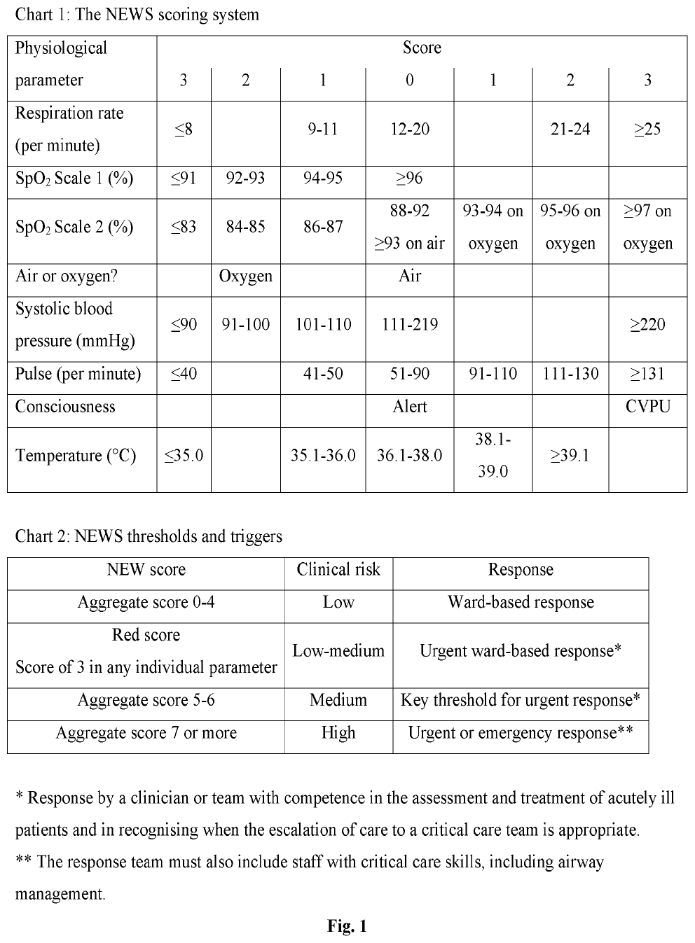

The National Early Warning Score is a tool developed by the Royal College of Physicians. At present, NEWS2, released in 2017, is the latest version. FIG. 1 shows NEWS2 charts used for the National Early Warning Score. The National Early Warning Score may be periodically updated. One of ordinary skill in the art will be able to access and apply the current version of the National Early Warning Score.

### Example 1

Protocol

| Title | Open-label, pharmacokinetic, pharmacodynamic, | | |
|---|---|---|---|
| | ascending dose, safety and tolerability lead-in followed | | |
| | by a single-center, randomized, placebo-controlled, | | |
| | double-blind, adaptive, safety and efficacy, pilot study of | | |
| | Trans Sodium Crocetinate (TSC) in SARS-CoV-2 | | |
| | Infected Subjects | | |
| Indication | Treatment of hypoxemia associated with respiratory | | |
| | SARS-CoV-2 infection | | |
| Study | Open-label, pharmacokinetic, pharmacodynamic, | | |
| Design | ascending dose, safety and tolerability lead-in followed | | |
| | by a single-center, randomized, placebo-controlled, | | |
| | double-blind, adaptive, safety and efficacy pilot | | |
| Study | Hospitalized patients with confirmed SARS-CoV-2 | | |
| Population | infection and hypoxemia, defined as SpO₂ < 94% on | | |
| | room air or requiring supplemental oxygen, WHO | | |
| | ordinal scale scores of 3, 4 or 5 | | |
| Objectives | Lead-In PK/PD and Dose Selection | | |
| | Determine the safety and tolerability of TSC when | | |
| | administered four times per day for up to 5 days for each | | |
| | of the doses to be studied. | | |
| | Determine the relative degree of improvement by TSC | | |
| | dose in blood oxygenation following treatment with TSC | | |
| | as measured by the SpO₂:FiO₂ (S:F) ratio via recorded | | |
| | continuous pulse oximetry. | | |
| | For pharmacodynamic and pharmacokinetic purposes | | |
| | determine blood oxygenation as measured by arterial | | |
| | oxygen partial pressure (PaO₂ in mmHg) to fractional | | |
| | inspired oxygen (FiO₂), the PaO₂:FiO₂ (P:F) ratio or | | |
| | alternatively the S:F ratio following TSC administration | | |
| | at each dose level with matching PK blood sampling. | | |
| | Determine the optimum, safe and tolerable biologic dose | | |
| | of TSC among the doses to be studied given four times | | |
| | per day (every 6 hours) for up to 5 days using the S:F | | |
| | ratio. | | |
| | Randomized pilot | | |
| | Determine the safety and efficacy of TSC administered | | |
| | at the selected optimum, safe and tolerable biologic dose | | |
| | four times per day (every 6 hours) for up to 15 days as | | |
| | compared to placebo. | | |
| Study | Lead-In PK/PD and Dose Selection | | |
| Overview | Each TSC dose will be administered as an IV bolus | | |
| | injection to subjects per dose level administered four | | |
| | times per day (every 6 hours) for up to 15 days (a | | |
| | minimum of 5 days). TSC dose levels will be studied. | | |
| | Subjects will be assigned to dose levels in ascending | | |
| | order. The dose range is as follows. | | |
| | 0.25 mg/kg TSC + Standard of Care | | |
| | 0.50 mg/kg TSC + Standard of Care | | |
| | 1.00 mg/kg TSC + Standard of Care | | |
| | 1.50 mg/kg TSC + Standard of Care | | |
| | 2.00 mg/kg TSC + Standard of Care | | |
| | 2.50 mg/kg TSC + Standard of Care | | |
| | As subjects complete 5 days of treatment they may | | |
| | continue at their assigned TSC dose four times per day | | |
| | (every 6 hours) for up to 15 days at the investigator’s | | |
| | discretion. | | |
| | At the completion of the lead-in the Safety Monitoring | | |
| | Committee (SMC) will examine the resultant safety and | | |
| | blood oxygenation (S:F) data for all subjects. | | |
| | Randomized pilot | | |
| | Two arm, randomized pilot. TSC dosing will be at the | | |
| | selected optimum, safe and tolerable biologic dose with | | |
| | an active to placebo ratio of 2:1 toward providing the | | |
| | maximum potential benefit to subjects. If two doses | | |
| | of TSC are to be studied in the randomized pilot the | | |
| | active to placebo ratio will be 2:2:1. Randomization will | | |
| | be stratified by disease severity, age and presence of pre- | | |
| | specified comorbidities. The treatment arms are as | | |
| | follows. | | |
| | TSC + Standard of Care | | |
| | Placebo + Standard of Care | | |
| | Each TSC dose will be administered as an IV bolus | | |
| | injection 4 times per day (every 6 hours) for up to 15 | | |
| | days. | | |
| | Subjects randomized to placebo will receive an IV bolus | | |
| | injection of an equivalent volume by patient weight of | | |
| | Normal Saline four times per day (every 6 hours) for up | | |
| | to 15 days. | | |
| | All study drug administration will be performed by | | |
| | unblinded medical staff. | | |
| | Blood oxygenation will be measured via recorded | | |
| | continuous pulse oximetry and the S:F ratio calculated. | | |
| | Provided that an arterial line is established, serial arterial | | |
| | blood gas measurements will be collected and recorded | | |
| | ~2 minutes prior to TSC administration and at 1 minute, | | |
| | 30 minutes, 1.5, 3, 6 hours post the first TSC | | |
| | administration. The calculated P:F ratio for the same | | |
| | time points will be recorded. This procedure will occur | | |
| | only once per subject per TSC dose level at Day 1 only. | | |
| | Alternatively, the S:F ratio may be used as the measure | | |
| | of blood oxygenation with recording of the S:F ratio at | | |
| | the corresponding timepoints. | | |
| | Subjects will be assessed daily while hospitalized. | | |
| | Discharged subjects will be asked to attend study visits | | |
| | at days 15, 29 and 60. | | |
| | All subjects will undergo safety and efficacy assessments | | |
| | including laboratory assays, blood sampling on days 1 | | |
| | through day 15 (while hospitalized) and day 29 by return | | |
| | clinic visit or if still hospitalized. | | |
| | All subjects whether a part of the lead-in phase or | | |
| | randomized pilot will be assessed for survival, serious | | |
| | adverse events and adverse events by requested return to | | |
| | the clinic on Day 60. | | |
| | Clinical Support | | |
| | At each study day, while hospitalized, the following | | |
| | measures of clinical support should be assessed and | | |
| | recorded. | | |
| | Hospitalization | | |
| | Oxygen requirement | | |
| | Non-invasive mechanical ventilation (via mask) | | |
| | Mechanical ventilator requirement (via endotracheal tube | | |
| | or tracheostomy) | | |
| | ECMO requirement | | |
| | WHO Ordinal Scale | | |
| | The WHO 9-point ordinal scale assessment will be the | | |
| | first assessment of the subject’s clinical status each day. | | |
| | Each day, the worst score for the previous day will be | | |
| | recorded (i.e. on day 3, the score for day 2 is recorded as | | |
| | day 2). The ordinal scale is as follows. | | |
| | Patient State | Descriptor | Score |
| | Uninfected | No clinical or | 0 |
| | | virological evidence | |
| | | of infection | |
| | Ambulatory | No limitation of | 1 |
| | | activities | |
| | | Limitation of | 2 |
| | | activities | |
| | Hospitalized | Hospitalized, no | 3 |
| | Mild Disease | oxygen therapy | |
| | | Oxygen by mask or | 4 |
| | | nasal prongs | |
| | Hospitalized | Non-invasive | 5 |
| | Severe Disease | ventilation or high- | |
| | | flow oxygen | |
| | | Intubation and | 6 |
| | | mechanical | |
| | | ventilation | |
| | | Ventilation + | 7 |
| | | additional organ | |
| | | support - | |
| | | vasopressors, RRT | |
| | | (renal replacement | |
| | | therapy), ECMO | |
| | | (extracorporeal | |
| | | membrane | |
| | | oxygenation) | |
| | Dead | Death | 8 |

| | NEW Score | |
|---|---|---|
| | The National Early Warning Score (NEWS) will be used | |
| | to record the seven key physiological parameters that | |
| | form the basis of the scoring system. | |
| | 1. Respiration rate | |
| | 2. Oxygen saturation (e.g., SpO₂) | |
| | 3. Air or oxygen | |
| | 4. Systolic blood pressure | |
| | 5. Pulse rate | |
| | 6. Level of consciousness or new confusion | |
| | 7. Temperature | |
| | The NEWS score will be recorded as a first assessment at | |
| | each study day using the colorized NEWS scoring system | |
| | and NEWS daily chart as a means of recording all 7 | |
| | parameters each day while hospitalized. | |
| | Glasgow Coma Scale | |
| | The Glasgow Coma Score will be performed daily until | |
| | hospital discharge and in accordance with the standards | |
| | established by the Institute of Neurological Sciences NHS | |
| | Greater Glasgow and Clyde. The Glasgow Coma Score | |
| | will be calculated individually and by addition of the total | |
| | points selected under each of the three components | |
| | including eye, verbal and motor. | |
| | Sequential Organ Failure Assessment (SOFA) | |
| | The Sequential Organ Failure Assessment (SOFA) Score | |
| | is a mortality prediction score that is based on the degree | |
| | of dysfunction of six organ systems. The score will be | |
| | calculated on admission and every 24 hours until | |
| | discharge using the worst parameters measured during the | |
| | prior 24 hours. | |
| Participant | An individual subject will complete the study in about 29 | |
| Duration | days, from screening/baseline at day −1 or 1 to follow-up | |
| | on day 29 ± 3 days. All subjects whether a part of the | |
| | lead-in phase or randomized pilot will be assessed for | |
| | survival, serious adverse events and adverse events by | |
| | requested return to the clinic on Day 60. | |
| Endpoints | Primary | |
| | Lead-In PK/PD | |
| | Serious Adverse Events/Adverse Events (Dose Limiting | |
| | Toxicity) | |
| | Randomized pilot | |
| | Time to recovery through Day 28, defined as time to | |
| | achieve (and maintain through Day 28) a WHO ordinal | |
| | COVID-19 severity scale score of 1, 2 or 3 with a | |
| | minimum 1-point improvement from baseline. | |
| | Secondary | |
| | WHO Ordinal severity scale: | |
| | Proportion of subjects with WHO ordinal severity scale | |
| | score of 6 or 7 at any time through Day 28 | |
| | Time to an improvement of one category (i.e., a 1-point | |
| | improvement) from baseline | |
| | Change from baseline in WHO scale score at days 2, 4, 7, | |
| | 10, 14 and 28, as a categorical improvement or worsening | |
| | Mean change in WHO ordinal severity scale score from | |
| | baseline through days 2, 4, 7, 10, 14 and 28 | |
| | National Early Warning Score (NEWS): | |
| | The time to discharge or to a NEWS of <2 and maintained | |
| | for 24 hours, whichever occurs first | |
| | Change from baseline through days 2, 4, 7, 10, 14 and 28 | |
| | in NEWS | |
| | Oxygenation: | |
| | Oxygenation free days in the first 28 days from start of | |
| | therapy | |
| | Incidence and duration of new oxygen use during the trial | |
| | Proportion on mechanical ventilation, ECMO, | |
| | noninvasive ventilation and high-flow nasal cannula | |
| | oxygen delivery and return to room air or baseline oxygen | |
| | requirement | |
| | Time to return to room air or baseline oxygen requirement | |
| | Days on extracorporeal membrane oxygenation (ECMO) | |
| | Blood oxygenation by recorded continuous pulse | |
| | oximetry (SpO₂:FiO₂ ratio) | |
| | Blood oxygenation by serial arterial blood gas | |
| | measurements collected prior to the first dose of TSC and | |
| | at 1 minute, 10 minutes, 30 minutes, 1.5 hours, 3 hours | |
| | and 6 hours post TSC administration by calculated | |
| | PaO₂:FiO₂ ratios | |
| | Mechanical Ventilation: | |
| | Ventilator free days in the first 28 days (to day 29). | |
| | Incidence and duration of new mechanical ventilation use | |
| | during the trial | |
| | Hospitalization | |
| | Hospital length of stay by Day 29 | |
| | ICU length of stay by Day 29 | |
| | Mortality | |
| | 15-day mortality | |
| | 28-day mortality | |
| | All-cause mortality at day 29 | |
| | In hospital mortality | |
| | Mortality at Day 60 | |
| | Other | |
| | Glasgow Coma Score | |
| | Sequential Organ Failure Assessment (SOFA) Score at | |
| | baseline, 24 and 48 hours, Day 7, Day 15 | |
| | 28-day vasopressor free days | |
| | Development of acute kidney injury (as defined by AKIN | |
| | criteria) | |
| | 28-day new renal replacement therapy (RRT) free days | |
| | (excluding patients on chronic HD) | |
| | Proportion of patients alive and free of respiratory failure | |
| | by Day 28 defined as at least one of the following: | |
| | Endotracheal intubation and mechanical ventilation | |
| | Oxygen delivered by high-flow nasal cannula (heated, | |
| | humidified oxygen delivered via reinforced nasal | |
| | cannula at flow rates >20 L/min with fraction of | |
| | delivered oxygen ≥0.5) | |
| | Noninvasive positive pressure ventilation | |
| | Extracorporeal membrane oxygenation | |
| | Clinical diagnosis of respiratory failure with initiation | |
| | of none of these measures only when clinical decision | |
| | making is driven solely by resource limitation | |
| | Safety | |
| | Cumulative incidence of serious adverse events (SAEs) to | |
| | Day 60 | |
| | Cumulative incidence of Grade 3 and 4 adverse events | |
| | (AEs) to Day 60 | |
| | Discontinuation or temporary suspension of study drug | |
| | injections (for any reason). | |
| | Changes in white cell count, haemoglobin, platelets, | |
| | creatinine, glucose, total bilirubin, ALT, and AST on days | |
| | 1, 3, 5, 8, 11 (while hospitalized); and Day 15 and 29 (if | |
| | able to return to clinic or still hospitalized | |
| | Death | |
| | DVT (deep vein thrombosis)/PE (pulmonary embolism) | |
| | Nervous system disorders | |
| | Respiratory (acute respiratory failure, cough, pneumonia) | |
| | Angina | |
| | Infections including sepsis | |
| | Injection site reactions | |
| | Drug hypersensitivity | |
| Inclusion | 1. Hospitalized subjects with confirmed SARS-CoV-2 | |
| Criteria | infection and hypoxemia, defined as SpO₂ < 94% on | |
| | room air or requiring supplemental oxygen | |
| | 2. Laboratory-confirmed SARS-CoV-2 infection as | |
| | determined by PCR, or other commercial or public health | |
| | assay in any specimen <72 hours prior to enrollment. | |
| | 3. WHO ordinal scale score of 3, 4 or 5 at baseline | |
| | 4. Male or non-pregnant female adult ≥18 years of age at | |
| | time of enrolment. | |
| | 5. Subject (or legally authorized representative) provides | |
| | written informed consent prior to initiation of any study | |
| | procedures. | |
| | 6. Understands and agrees to comply with planned study | |
| | procedures. | |
| | 7. Agrees to the collection of venous blood per protocol. | |
| | 8. Illness of any duration | |
| | 9. Women of childbearing potential must have a negative | |
| | blood pregnancy test at the screening/baseline visit (Day | |
| | 1) and agree to use a double method of birth control | |
| | through 30 days after the last dose of study drug. | |
| Exclusion | 1. Intubated and mechanically ventilated at baseline | |
| Criteria | 2. Receiving extracorporeal membrane oxygenation | |
| | (ECMO) at baseline | |
| | 3. Severe organ dysfunction (SOFA score > 10) | |
| | 4. Patient or LAR unable to provide written informed | |
| | consent | |
| | 5. ALT/AST >3 times, e.g., >5 times, the upper limit of | |
| | normal or serum bilirubin >1.5 times the upper limit of | |
| | normal. | |
| | 6. Stage 3 (eGFR (estimated glomerular filtration rate) by | |
| | Modification of Diet in Renal Disease (MDRD) formula | |
| | severe chronic kidney disease or requiring dialysis (i.e., | |
| | eGFR <30) or eGFR by MDRD formula <30 | |
| | mL/min/1.73 m² or on dialysis | |
| | 7. Pregnancy or breast feeding. | |
| | 8. Anticipated transfer to another hospital which is not a | |
| | study site within 72 hours. | |
| | 9. Allergy to any study medication | |
| PK/PD | The pharmacokinetics and pharmacodynamics of TSC | |
| | will be assessed via an ascending dose lead-in prior to | |
| | initiating the randomized trial. Subjects will be studied at | |
| | 4 doses between 0.25 mg/kg and 1.5 mg/kg and at 2.0 | |
| | mg/kg and 2.5 mg/kg administered as an IV bolus | |
| | injection 4 times per day (every 6 hours) for up to 5 days. | |
| | Subjects will be assigned (not randomized between | |
| | treatments) at baseline. Blood samples for | |
| | pharmacokinetic purposes will be collected only at | |
| | baseline (Day 1) for each subject at each dose level. | |
| | TSC dose | Duration (days) |
| | 0.25 mg/kg | 5 |
| | 0.50 mg/kg | 5 |
| | 1.00 mg/kg | 5 |
| | 1.50 mg/kg | 5 |
| | 2.00 mg/kg | |
| | 2.50 mg/kg | |

| | |
|---|---|
| | The PK blood sampling plan is as follows. |
| | Baseline (Day 1) |
| | pre-dose (~2 minutes prior to injection) |
| | 1 minute post end of injection (±1 minute) |
| | 30 minutes (± 1 min) |
| | 1.5 hours (± 2 min) |
| | 3 hours (± 5 min) |
| | 6 hours (±10 min) |
| | 24 hours (± 1 hour) |
| | 48 hours (± 2 hours) |
| | 96 hours (± 2 hours) |
| | Provided that an arterial line is established, blood |
| | oxygenation data by serial arterial blood gas and the |
| | calculated PaO₂:FiO₂ ratios will form the means of a |
| | pharmacokinetic/pharmacodynamic assessment. |
| | Blood oxygenation data by recorded continuous pulse |
| | oximetry (SpO₂:FiO₂ ratio) will serve as an alternate |
| | source in the absence of arterial blood gas data. |
| Statistical | Time to recovery through Day 28 is the primary efficacy |
| | endpoint for the randomized part of the study. Subjects |
| | enter the study with a WHO COVID-19 ordinal severity |
| | scale score of 3, 4 or 5. To meet the definition of |
| | recovery, a subject must achieve a WHO severity score of |
| | 1, 2 or 3 and have an improvement of at least 1 point, |
| | maintained through the Day 28. Time to recovery will be |
| | calculated from day of randomization to day of recovery. |
| | Subjects who have not recovered by Day 29 evaluation |
| | will be censored at Day 28. A stratified log-rank test will |
| | be used to test the primary endpoint of time to recovery. |
| | The key secondary endpoints in order of importance are |
| | hospital length of stay by Day 29 (so through Day 28), |
| | proportion of subjects with WHO severity score of 6 or 7 |
| | at any time through Day 28, and all-cause mortality at |
| | Day 29 (so through Day 28). A hierarchical approach to |
| | the testing of these endpoints will be performed if the |
| | primary efficacy endpoint is statistically significant. |

### Study Rationale

COVID-19 is a respiratory disease caused by a novel coronavirus (SARS-CoV-2) associated with substantial morbidity and mortality. This clinical trial is designed to evaluate the safety and efficacy of trans sodium crocetinate (TSC) to improve oxygenation in SARS-CoV-2 infected patients with hypoxemia as a means of mitigating the unfortunate progression to acute respiratory distress syndrome (ARDS) and systemic organ injury.

### Study Overview

The trial is composed of an open-label, pharmacokinetic, pharmacodynamic, ascending dose, safety and tolerability lead-in study to a single-center, randomized, placebo-controlled, double-blind, adaptive, safety and efficacy pilot study of TSC in SARS-CoV-2 infected patients with hypoxemia. The study includes assessment of blood oxygenation via continuous pulse-oximetry (SpO₂) with calculation of the SpO₂:FiO₂ ratios (S:F ratio). The lead-in phase as well as the randomized phase also include serial blood gas (ABG) measurements on Day 1 prior to TSC administration and at 1 minute, 30 minutes, 3 hours and 6 hours with matching pharmacokinetic blood sampling.

### Study Objectives

### Lead-In PK/PD and Dose Selection

1. Determine the safety and tolerability of TSC when administered four times per day (every 6 hours) for up to 5 days for each of the doses to be studied.
2. Determine the relative degree of improvement by TSC dose in blood oxygenation following treatment with TSC as measured by the SpO₂:FiO₂ (S:F) ratio via continuous pulse oximetry.
3. For pharmacodynamic and pharmacokinetic purposes determine blood oxygenation as measured by arterial oxygen partial pressure (PaO₂ in mmHg) to fractional inspired oxygen (FiO₂), the PaO₂:FiO₂ (P:F) ratio or S:F ratio following the TSC administration at each dose level with matching PK blood sampling.
4. Determine the optimum, safe and tolerable biologic dose of TSC among the doses to be studied given four times per day (every 6 hours) for up to 5 days using the S:F ratio.

### Randomized Pilot

1. Determine the safety and efficacy of TSC administered at the optimum, safe and tolerable biologic dose four times per day (every 6 hours) for up to 15 days as compared to placebo.
2. Demonstrate that TSC is not associated with an increased occurrence of serious adverse events in COVID-19 patients. The study endpoint analysis will compare the frequency of SAEs in the TSC and placebo groups.
3. Demonstrate that treatment with TSC is not associated with increases in any organ-specific classes of serious adverse events or increased mortality.

### Primary Endpoints

### Lead-In PK/PD

Serious adverse events/Adverse events (Dose Limiting Toxicity), with the exception of pulmonary events in the CTCAE that are known complications of SARS-CoV-2 infection: ARDS, Cough, Dyspnea, Hypoxia, Pneumonitis, Pulmonary Edema, Respiratory Failure, or Respiratory, Thoracic and Mediastinal disorders.
   Randomized pilot
Time to recovery through Day 28, defined as time to achieve (and maintain through Day 28) a WHO ordinal severity scale score of 1, 2 or 3 with a minimum 1-point improvement from baseline

### Secondary Endpoints

WHO ordinal severity scale score:
Proportion of subjects with WHO ordinal severity scale score of 6 or 7 at any time through Day 28
Time to an improvement of one category (i.e., a 1-point improvement) from baseline
Change from baseline in WHO ordinal severity scale score at days 2, 4, 7, 10, 14 and 28, as a categorical improvement or worsening
Mean change in WHO ordinal severity scale score from baseline through days 2, 4, 7, 10, 14 and 28

### National Early Warning Score (NEWS):

The time to discharge or to a NEWS of ≤2 and maintained for 24 hours, whichever occurs first
Change from baseline through days 2, 4, 7, 10, 14 and 28 in NEWS

### Oxygenation:

Oxygenation free days in the first 28 days from start of therapy
Incidence and duration of new oxygen use during the trial
Proportion on mechanical ventilation, ECMO, noninvasive ventilation and high-flow nasal cannula oxygen delivery and return to room air or baseline oxygen requirement
Time to return to room air or baseline oxygen requirement
Days on extracorporeal membrane oxygenation (ECMO)
Blood oxygenation by recorded continuous pulse oximetry (SpO₂:FiO₂ ratio)
Blood oxygenation by serial arterial blood gas measurements collected prior to the first dose of TSC and at 1 minute, 30 minutes, 1.5 hours, 3 hours and 6 hours post TSC administration by calculated PaO₂:FiO₂ ratios
Durability of blood oxygenation via SpO₂:FiO₂ ratios

### Mechanical Ventilation:

Ventilator free days in the first 28 days (to day 29)
Incidence and duration of new mechanical ventilation use during the trial

### Hospitalization

Hospital length of stay by Day 29
ICU length of stay by Day 29

### Mortality

15-day mortality
28-day mortality
All-cause mortality at day 29
In hospital mortality
Mortality at Day 60

### Other

Glasgow Coma Score
Sequential Organ Failure Assessment (SOFA) Score at baseline, 24 and 48 hours, Day 7, Day 15
28-day vasopressor free days
Development of acute kidney injury (as defined by AKIN criteria)
28-day new renal replacement therapy (RRT) free days (excluding patients on chronic HD)
Proportion of patients alive and free of respiratory failure by Day 28 defined as at least one of the following:
   Endotracheal intubation and mechanical ventilation
   Oxygen delivered by high-flow nasal cannula (heated, humidified oxygen delivered via reinforced nasal cannula at flow rates >20 L/min with fraction of delivered oxygen ≥0.5)
   Noninvasive positive pressure ventilation
   Extracorporeal membrane oxygenation
   Clinical diagnosis of respiratory failure with initiation of none of these measures only when clinical decision making is driven solely by resource limitation

### Safety

Cumulative incidence of serious adverse events (SAEs) to Day 60
Cumulative incidence of Grade 3 and 4 adverse events (AEs) to Day 60
Discontinuation or temporary suspension of study drug injections (for any reason)
Changes in white cell count, haemoglobin, platelets, creatinine, glucose, total bilirubin, ALT, and AST from day 1 through day 15 (while hospitalized); and day 29 (if able to return to clinic or still hospitalized)
Death
DVT/PE
Nervous system disorders
Respiratory (acute respiratory failure, cough, pneumonia)
Angina
Infections including sepsis
Injection site reactions
Drug hypersensitivity

### Dosing Regimen

### Lead-In PK/PD

Each TSC dose will be administered as an IV bolus injection to subjects per dose level administered four times per day (every 6 hours) for up to 5 days. TSC dose levels will be studied. Subjects will be assigned to dose levels in ascending order. The dose range is as follows.
0.25 mg/kg TSC+Standard of Care
0.50 mg/kg TSC+Standard of Care
1.00 mg/kg TSC+Standard of Care
1.50 mg/kg TSC+Standard of Care
2.00 mg/kg TSC+Standard of Care
2.50 mg/kg TSC+Standard of Care

This study will utilize an ascending dose scheme, starting at 0.25 mg/kg.

As subjects complete 5 days of treatment they may continue at their assigned TSC dose for up to 15 days at the investigator's discretion.

At the completion of the lead-in the SMC will examine the safety and blood oxygenation (S:F) data for all subjects and determine the optimum, safe and tolerable dose of TSC for use in the pilot study.

### Randomized Pilot

TSC dosing will be at the selected optimum, safe and tolerable biologic dose with an active to placebo ratio of 2:1 or 2:2:1 if two TSC doses are to be studied. The treatment arms are as follows.
TSC+Standard of Care
Placebo+Standard of Care

Each TSC dose will be administered as an IV bolus injection 4 times per day (every 6 hours) for up to 15 days.

Subjects randomized to placebo will receive an IV bolus injection of Normal Saline at a volume which is matched to the volume that they would receive if they were receiving TSC, 4 times per day (every 6 hours) for up to 15 days.

All study drug administration will be performed by unblinded medical staff.

Blood oxygenation will be measured via recorded continuous pulse oximetry and the S:F ratio calculated.

For both the lead-in phase and randomized phase, provided that an arterial line is established, serial arterial blood gas measurements will be collected and recorded prior to TSC administration and at 1 minute, 30 minutes, 3 hours and 6 hours post TSC administration and the P:F ratio calculated, but only once per subject per TSC dose level. Alternatively, the S:F ratio will be used.

Subjects will be assessed daily while hospitalized. Discharged subjects will be asked to attend study visits at days 15, 29 and 60. All subjects whether a part of the lead-in phase or randomized pilot will be assessed for survival, serious adverse events and adverse events by requested return to the clinic on Day 60.

All subjects will undergo safety and efficacy assessments including laboratory assays, blood sampling on day 1 through day 15 (while hospitalized) and day 29 by return clinic visit or if still hospitalized.

### Clinical Support

For both the lead-in phase and randomized phase, at each study day, while hospitalized, the following measures of clinical support should be assessed and recorded.
Hospitalization
Oxygen requirement
Non-invasive mechanical ventilation (via mask)
Mechanical ventilator requirement (via endotracheal tube or tracheostomy)
ECMO requirement

### WHO Ordinal Severity Scale Score

For both the lead-in phase and randomized phase, the WHO 9-point ordinal scale assessment will the first assessment of the subject's clinical status each day. Each day, the worst score for the previous day will be recorded (i.e. on day 3, the score for day 2 is recorded as day 2). The ordinal scale is as follows.

| Patient State | Descriptor | Score |
|---|---|---|
| Uninfected | No clinical or | 0 |
| | virological evidence | |
| | of infection | |
| Ambulatory | No limitation of | 1 |
| | activities | |
| | Limitation of | 2 |
| | activities | |
| Hospitalized | Hospitalized, no | 3 |
| Mild Disease | oxygen therapy | |
| | Oxygen by mask or | 4 |
| | nasal prongs | |
| Hospitalized | Non-invasive | 5 |
| Severe Disease | ventilation or high- | |
| | flow oxygen | |
| | Intubation and | 6 |
| | mechanical | |
| | ventilation | |
| | Ventilation + | 7 |
| | additional organ | |
| | support—pressors, | |
| | RRT, ECMO | |
| Dead | Death | 8 |

### NEWS Score

For both the lead-in phase and randomized phase, the National Early Warning Score (NEWS) will be used each day to record the seven key physiological parameters that form the basis of the scoring system.
1. Respiration rate
2. Oxygen saturation (e.g., SpO₂)
3. Air or oxygen
4. Systolic blood pressure
5. Pulse rate
6. Level of consciousness or new confusion*
7. Temperature
   The NEWS score will be recorded as a first assessment each study day using the colorized NEWS Scoring System and NEWS Daily Chart as a means of record keeping.

### Study Population

For both the lead-in phase and randomized phase, the study population will consist of hospitalized patients with confirmed SARS-CoV-2 infection and hypoxemia, defined as SpO₂<94% on room air or requiring supplemental oxygen, WHO ordinal scale 3, 4 or 5 and further characterized by inclusion and exclusion criteria of this protocol.

For the randomized phase, the population to be analyzed is the Intention-to-Treat (ITT) dataset (i.e., all randomized participants). We do not anticipate subject dropout for the primary outcome, and there will be no ‘drop-in’ of usual care participants receiving TSC. The safety dataset will include all randomized participants.

### Inclusion Criteria

1. Hospitalized subjects with confirmed SARS-CoV-2 infection and hypoxemia, defined as SpO₂<94% on room air or requiring supplemental oxygen.
2. Laboratory-confirmed SARS-CoV-2 infection as determined by PCR, or other commercial or public health assay in any specimen <72 hours prior to enrollment.
3. WHO ordinal scale score of 3, 4 or 5 at baseline.
4. Male or non-pregnant female adult ≥18 years of age at time of enrollment.
5. Subject (or legally authorized representative) provides written informed consent prior to initiation of any study procedures.
6. Understands and agrees to comply with planned study procedures.
7. Agrees to the collection of venous blood per protocol.
8. Illness of any duration.
9. Women of childbearing potential must have a negative blood pregnancy test at the screening/baseline visit (Day 1) and agree to use two forms of birth control through 30 days after the last dose of study drug.

### Exclusion Criteria

1. Intubated and mechanically ventilated at baseline.
2. Receiving extracorporeal membrane oxygenation (ECMO) at baseline.
3. Severe organ dysfunction (SOFA score >10).
4. Patient or LAR unable to provide written informed consent.
5. ALT/AST>3 times, e.g., >5 times, the upper limit of normal.
6. Stage 3 (eGFR by MDRD) severe chronic kidney disease or requiring dialysis (i.e., eGFR<30) or eGFR by MDRD formula <30 mL/min/1.73 m².
7. Pregnancy or breast feeding.
8. Anticipated transfer to another hospital which is not a study site within 72 hours.
9. Allergy to any study medication
10. Moribund patient not expected to survive 24 hours.

### Randomization

Subjects participating in the open-label, PK/PD, ascending dose, safety and tolerability lead-in will be assigned to treatment with TSC (not randomized) in ascending dose fashion.

Subjects participating in the single-center, randomized, placebo-controlled, double-blind, adaptive, safety and efficacy pilot will be randomized to the selected optimum, safe and tolerable biologic dose of TSC or placebo with an active to placebo ratio of 2:1 or 2:2:1 if two TSC doses are to be studied. Randomization to treatment will be stratified by the following factors assessed at randomization:
Disease severity (WHO ordinal scale 3 vs 4 or 5)
Age at enrollment (<60 years vs ≥60 years of age)
Comorbidities (presence of any of the following: hypertension, diabetes or immune-related disease vs absence of all)

The identified person administering study drug will be unblinded given the orange-red coloration of the reconstituted TSC solution compared to Normal Saline placebo. Unblinded personnel will play no role in making patient assessments.

The lead-in PK/PD dose selection phase is not randomized whereas the follow-on pilot study is randomized. Both phases require four times a day (every 6 hour) dosing for up to 15 days.

### Study Drug

### Trans Sodium Crocetinate (TSC)

Trans sodium crocetinate (TSC) will be administered intravenously as an IV bolus. The concentration of the reconstituted drug is 20 mg/mL.

TSC is dosed based on the patient's baseline weight, obtained on the day of screening, on a milligram per kilogram basis.

### Placebo

For the randomized phase, subjects randomized to placebo will receive an IV bolus injection of Normal Saline at a volume which is matched to the volume that they would receive if they were receiving TSC, four times per day (every 6 hours) for up to 15 days.

### Concomitant Therapy

Subjects enrolled in this trial may receive any conventional treatment at the discretion of their attending physicians.

Therapy with antivirals including remdesivir or lopinavir/ritonavir (Kaletra®) or other therapeutic agents (e.g., corticosteroids) prior to enrollment in this trial are permitted.

If the local standard of care per written policies or guidelines (i.e., not just an individual clinician decision) includes remdesivir, lopinavir/ritonavir (Kaletra®) or other agents, then continuing these during the study is permitted, but may require additional safety monitoring by the site.

All medications taken in the 7 days prior to the first dose of study drug will be captured in the electronic case report form (eCRF). After the first dose of study drug, concomitant medications to be captured in the eCRF will be vasopressors and any medication given to specifically target COVID-19 (e.g., antivirals and corticosteroids).

### Baseline Evaluation (Day −1/Day 1)

Information commonly collected and recorded in source documents at the time of hospital admission for hypoxemia associated with SARS-CoV-2 infection can be used to qualify a subject for the study and hence, need not be repeated.

Following completion of the informed consent process the study coordinator will record the following from source documents or perform the following.
Demography
Medical History
Vital signs (heart rate, blood pressure, respiratory rate, temperature)
Full physical examination
12-Lead ECG
Oxygen saturation (with calculated SpO₂:FiO₂) by recorded continuous pulse oximetry
Arterial blood gas measurement (with calculated PaO₂:FiO₂), if monitored
WHO 9-point ordinal severity scale score
NEWS score (using the colorized NEWS scoring system and NEWS Daily Chart)
Glasgow Coma Score (per supplied NHS Greater Glasgow & Clyde)
Sequential Organ Failure Assessment (SOFA)
   Laboratory data that will be captured at baseline includes:
Complete blood count (CBC)
Basic metabolic panel (BMP)
Creatine kinase (CK), creatine kinase muscle-brain (CK-MB), glutamate dehydrogenase (GLDH), troponin
Liver function test (LFT) or hepatic panel
Coagulation panel
Serology panel
Urinalysis
Laboratory confirmation of SARS-CoV-2 infection
Blood pregnancy test for women of child bearing potential
SARS-CoV-2 IgM and IgG antibodies
SARS-CoV-2 viral load
Immunological assessments
   Imaging data at baseline will include:
Chest X-Ray (CT Scan)

Subjects meeting all inclusion/exclusion criteria and who have completed the informed consent process may be enrolled in the study.

The unblinded bedside nurse and physician who are delegated, may then perform the following:
Schedule all PK blood sample collection times from pre-dose through 96 hours
Vital signs taken prior to every dose of study drug
Collect pre-dose PK blood sample (time zero ˜2 minutes prior to study drug administration)
Administer study drug (active or placebo) by IV bolus injection
Record the end of injection time (hour/minutes using appropriate timepiece)
Perform the PK blood sample collections as follows
   Pre-dose PK blood sample collection (˜2 minutes prior to study drug)
   1 minute post the end of injection (±1 minute sec)
   30 minutes post the end of injection (±1 minute)
   1.5 hours post end of injection (±2 minutes)
   3.0 hours post end of injection (+5 minutes)
   6.0 hours post end of injection (+10 minutes)
Record oxygen saturation (SpaO₂) from the continuous recorded pulse oximetry at the same timepoints
   Pre-dose PK blood sample collection (˜2 minutes prior to study drug)
   1 minute post the end of injection (±1 minute)
   30 minutes post the end of injection (±1 minute)
   1.5 hours post end of injection (±2 minutes)
   3.0 hours post end of injection (±5 minutes)
   6.0 hours post end of injection (±10 minutes)
   Calculate and record the SpaO₂:FiO₂ ratio for each time point
Alternatively record oxygenation by arterial blood gas measurements (PaO₂), if ABG is monitored, at the same timepoints
   Additional PK blood sample collections
24 hours post end of initial injection (±1 hour)
48 hours post end of initial injection (±2 hours)
96 hours post end of initial injection (±2 hours)
   To be performed from Day 2 to the earlier of hospital discharge or Day 29
Vital signs will be taken prior to every dose of study drug
A targeted physical exam will conducted each day while the patient is hospitalized.
A 12-lead ECG will be performed at Days 7, 14, 21 and 28
Following the laboratory confirmation of SARS-CoV-2 infection at the Screening/Baseline (Day −1/Day 1) visit, SARS-CoV-2 IgM and IgG antibodies and SARS-CoV-2 viral load will be assessed at Days 7, 14, 21, and 28
Oxygen saturation (SpO₂:FiO₂) by continuous recorded pulse oximetry daily
Arterial blood gas measurements (PaO₂:FiO₂), if ABG monitored
Non-invasive mechanical ventilation (via mask); in liters/minute, if any, or Mechanical ventilator requirement (via endotracheal tube or tracheostomy); current ventilatory settings, if applicable, daily
WHO ordinal severity scale score, daily
NEWS (using the colorized NEWS scoring system and NEWS Daily Chart), daily
Glasgow Coma Score (per supplied NHS Greater Glasgow & Clyde), daily
Sequential Organ Failure Assessment (SOFA), daily
AKIN classification, daily
   To be performed on Days 2 through Day 15, and 29 (and additional days when hospitalized)
Laboratory data
   Complete blood count (CBC)
   Basic metabolic panel (BMP)
   Liver function test (LFT) or hepatic panel
   Coagulation panel (Coag)
   Immunological assessments (Imm)
   Urinalysis
      All subjects whether a part of the lead-in phase or randomized pilot will be assessed for survival, serious adverse events and adverse events by requested return to the clinic on Day 60 (+10 days).
      Assessments at the time of hospital discharge or Day 29
      The following assessments will be recorded in the case report form by review of the source documents for each subject at the time of hospital discharge or Day 29.
      WHO 9-point Ordinal Severity Scale Score:
Time to an improvement of one category from admission on the WHO ordinal scale
Subject clinical status on the WHO ordinal scale at days 2, 4, 7, 10, 14 and 28
Mean change in the ranking on an ordinal scale from baseline to days 2, 4, 7, 10, 14 and 28

### National Early Warning Score (NEWS):

The time to discharge or to a NEWS of ≤2 and maintained for 24 hours, whichever occurs first
Change from baseline to days 2, 4, 7, 10, 14 and 28 in NEWS

### Oxygenation:

Oxygenation free days in the first 28 days (to day 29)
Incidence and duration of new oxygen use during the trial
Proportion on mechanical ventilation, ECMO, noninvasive ventilation and high-flow nasal cannula oxygen delivery and return to room air or baseline oxygen requirement
Time to return to room air or baseline oxygen requirement
Days on extracorporeal membrane oxygenation (ECMO)
Blood oxygenation by recorded continuous pulse oximetry (SpO₂:FiO₂ ratio)
Blood oxygenation by serial arterial blood gas measurements collected prior to the first dose of TSC and at 1 minute, 30 minutes, 1.5 hours, 3 hours and 6 hours post TSC administration by calculated PaO₂:FiO₂ ratios

### Mechanical Ventilation:

Ventilator free days in the first 28 days (to day 29).
Incidence and duration of new mechanical ventilation use during the trial

### Hospitalization

Hospital length of stay by Day 29
ICU length of stay by Day 29

### Mortality

15-day mortality
28-day mortality
All-cause mortality at Day 29
In hospital mortality
Mortality by Day 60

### Other

Glasgow Coma Score
Sequential Organ Failure Assessment (SOFA) Score at baseline, 24 and 48 hours, Day 7, Day 15
28-day vasopressor free days
Development of acute kidney injury (as defined by AKIN criteria)
28-day new renal replacement therapy (RRT) free days (excluding patients on chronic HD)
Proportion of patients alive and free of respiratory failure by Day 28 defined as at least one of the following:
   Endotracheal intubation and mechanical ventilation
   Oxygen delivered by high-flow nasal cannula (heated, humidified oxygen delivered via reinforced nasal cannula at flow rates >20 L/min with fraction of delivered oxygen ≥0.5)
   Noninvasive positive pressure ventilation
   Extracorporeal membrane oxygenation
   Clinical diagnosis of respiratory failure with initiation of none of these measures only when clinical decision making is driven solely by resource limitation

### Safety

Serious adverse events (SAEs) day Day 60
Grade 3 and 4 adverse events (AEs) Day 60
Discontinuation or temporary suspension of study drug injections (for any reason).
Changes in white cell count, haemoglobin, platelets, creatinine, glucose, total bilirubin, ALT, and AST from day 1 through day 15 (while hospitalized); and day 29 (if able to return to clinic or still hospitalized)
Death
DVT/PE
Nervous system disorders
Respiratory (acute respiratory failure, cough, pneumonia)
Angina
Infections including sepsis
Injection site reactions
Drug hypersensitivity

### Analysis of the Primary Efficacy Endpoint

Time to recovery through Day 28 is the primary efficacy endpoint for the randomized part of the study. Subjects enter the study with a WHO COVID-19 ordinal severity scale score of 3, 4 or 5. To meet the definition of recovery, a subject must achieve a WHO severity score of 1, 2, or 3 and have an improvement of at least 1 point, maintained through the Day 28. In other words, subjects who enter the study with a baseline WHO scale score of 3 and improve to (and maintain to the Day 29 evaluation) a score of 1 or 2 have met the definition of recovery. Subjects who enter the study with a baseline WHO scale score of 4 or 5 and improve to (and maintain to Day 29 evaluation) a score of 1, 2 or 3 have met the definition of recovery. Time to recovery will counted from day of randomization to day of recovery (date of recovery−date of randomization+1).

Note that the evaluation that is performed on Day 29 is an assessment of the subject's WHO severity score of the previous day (Day 28).

### Glasgow Coma Score (GCS)

The Glasgow Coma Score will be performed daily until hospital discharge and in accordance with the standards established by the Institute of Neurological Sciences NHS Greater Glasgow and Clyde. The Glasgow Coma Score will be calculated by addition of the total points selected under each of the three components including eye, verbal and motor. The score for eye opening, verbal response and best motor response will be recorded individually as well as the total score, daily. The procedure as well as the template for recording the Glasgow Coma Scale is described at the web site www.glasgowcomascale.org.

### Sequential Organ Failure Assessment (SOFA)

The Sequential Organ Failure Assessment (SOFA) Score is a mortality prediction score that is based on the degree of dysfunction of six organ systems. The score is calculated on admission and every 24 hours until hospital discharge using the worst parameters measured during the prior 24 hours. Individual system scores and total will be recorded as follows. SaO₂/FiO₂ data may be substituted when PaO₂/FiO₂ data is unavailable.

It will be readily apparent to those skilled in the art that numerous modifications and additions can be made to both the present compounds and compositions, and the related methods without departing from the invention disclosed.

What is claimed is:

1. A method of treating a patient (e.g., a human) in need thereof having a viral or bacterial induced respiratory disease with hypoxemia comprising administering a diffusion enhancing compound to said patient.

2. A method of prophylaxis and/or treatment of hypoxemia consequent to a viral or bacterial induced respiratory disease in a patient (e.g., a human) in need thereof comprising administering a diffusion enhancing compound to said patient.

3. A method of prophylaxis and/or treatment of acute respiratory distress syndrome associated with a viral or bacterial respiratory disease in a patient (e.g., a human) in need thereof comprising administering a diffusion enhancing compound to said patient, wherein said diffusion enhancing compound is TSC.

4. A method of prophylaxis and/or treatment of acute respiratory distress syndrome consequent to a viral or bacterial respiratory disease in a patient in need thereof comprising administering a diffusion enhancing compound to said patient.

5. A method of prophylaxis and/or treatment of multiple organ failure consequent to a viral or bacterial respiratory disease in a patient in need thereof comprising administering a diffusion enhancing compound to said patient.

6. A method as in any one of claims 1-5 wherein the disease is influenza.

7. A method as in any one of claims 1-5 wherein the disease is a corona virus infection.

8. A method as in any one of claims 1-5 wherein the disease is COVID-19.

9. A method as in any one of claims 1-5 wherein the disease is bacterial or viral pneumonia.

10. A method as in any one of claims 1-9 wherein said diffusion enhancing compound is a bipolar trans carotenoid salt.

11. A method as in any one of claims 1-10 wherein said diffusion enhancing compound is a bipolar trans carotenoid salt and is formulated with a cyclodextrin, e.g., gamma-cyclodextrin.

12. A method as in any one of claims 1-11 wherein said diffusion enhancing compound is TSC.

13. A method as in any one of claims 1-12 wherein the diffusion enhancing compound is administered IV or IM.

14. A method as in any one of claims 1-13 wherein the diffusion enhancing compound is TSC and is administered at a dose of 0.2-2 mg/kg.

15. A method as in any one of claims 1-14 wherein the diffusion enhancing compound is TSC and is administered at a dose of 0.25-1.5 mg/kg.

16. A kit comprising:
a) a container comprising a diffusion enhancing compound, and
b) instructions for using the diffusion enhancing compound to treat a patient having or suspected of having a viral or bacterial induced respiratory disease by administering the diffusion enhancing compound at a dose of 0.05-2.5 mg/kg.
